(19) United States
(12) Patent Application Publication
(10) Pub. No.: US 2024/0068018 A1
(43) Pub. Date: Feb. 29, 2024

(54) INTEGRATED MOLECULAR SENSOR DEVICE AND METHOD FOR MAKING SAME

(71) Applicant: DrinkSavvy, Inc., Brooklyn, NY (US)

(72) Inventors: Min Hu, Brooklyn, NY (US); Jacob Timothy Trevino, Union, NJ (US)

(21) Appl. No.: 18/455,812

(22) Filed: Aug. 25, 2023

Related U.S. Application Data
(60) Provisional application No. 63/373,704, filed on Aug. 26, 2022.

(51) Int. Cl. C12Q 1/6844

(52) U.S. Cl. CPC C12Q 1/6844

(57) ABSTRACT

A sensor and a method of use may include a structure comprising a plurality of walls that define a plurality of air gaps in the structure, wherein each wall of the plurality of walls may include a plurality of surfaces. The sensor may include a functional layer, wherein the functional layer may be coated on the plurality of walls, wherein the functional layer comprises an extraction component to extract an analyte of interest, at least one amplification initiator to amplify the analyte of interest after extraction, and a material coating the plurality of walls providing an initial surface energy for at least a portion of the plurality of surfaces of the plurality of walls. The initial surface energy of at least the portion of the plurality of surfaces of the plurality of walls provided by the material coating may change when the analyte of interest is present.

### RELATED CASES

This application claims the benefit of U.S. Provisional Application No. 63/373,704, filed on 26 Aug. 2022, the contents of which are all incorporated by reference.

### BACKGROUND

Nucleic acid based molecular tests are the most accurate diagnostic assays; however, they typically require multiple steps, including sample preparation, amplification, and detection, most often requiring the use of additional instrumentation, including centrifuges, thermocyclers, heating sources, plate readers, etc., which adds cost and delays results.

### SUMMARY

In one example implementation, a method for detecting an analyte of interest may include but is not limited to contacting a sensor with a sample, wherein the integrated sensor includes a plurality of walls that define a plurality of air gaps in the structure, wherein each wall of the plurality of walls includes a plurality of surfaces, and further includes a functional layer coated on the plurality of walls, and further includes a material coating the plurality of walls providing an initial surface energy for at least a portion of the plurality of surfaces of the plurality of walls. An analyte of interest may be extracted with the functional layer. The analyte of interest may be amplified with the functional layer. The initial surface energy of at least the portion of the plurality of surfaces of the plurality of walls may be changed when the analyte of interest is present. The sensor may transition between a first mode and a second mode based upon, at least in part, the initial surface energy change.

One or more of the following example features may be included. The functional layer may further comprise a binding material to bind to the analyte of interest after amplification in a solution phase. A fluid sample comprising the analyte of interest may be prevented from exiting the plurality of air gaps based upon, at least in part, the change in the initial surface energy due to the binding material binding amplicons in solution on at least the portion of the plurality of surfaces of the plurality of walls. A fluid sample comprising the analyte of interest may be prevented from exiting the plurality of air gaps after amplification in a solid phase based upon, at least in part, the change in the initial surface energy due to amplicons formed on at least the portion of the plurality of surfaces of the plurality of walls. Multiple amplification initiators may be immobilized on at least the portion of the plurality of walls in spatially separated zones to enable multiplexing detection. A concentration of the analyte of interest may be quantitatively determined based upon, at least in part, a percentage of the initial surface energy change. Each amplification initiator may have a variable base, and a matching base that initiates an elongation of the each amplification initiator that produces the percentage of the initial surface energy change indicating a single nucleotide polymorphism (SNP) site. The plurality of walls coated with the functional layer may be fabricated from one of a micropillar array and a nanopillar array. Extraction and amplification may be conducted on the functional layer via at least one of a solution phase amplification and a solid phase amplification mechanism. The analyte of interest may be one of a DNA sequence, a RNA sequence, a single nucleotide polymorphism and multiple nucleotide polymorphism. The analyte of interest may be a complementary DNA (cDNA). An RNA target may form a heteroduplex with a trigger DNA to initiate amplification. A short microRNA sequence may be extended by a splinted ligation. A poly(A) tail may be added to a short microRNA sequence. One of an amplicon and a reverse primer may be tagged with an additional component to enhance the change of the initial surface energy on at least the portion of the plurality of surfaces of the plurality of walls. The analyte of interest may be amplified by one of loop-mediated isothermal amplification (LAMP), helicase dependent amplification (HDA), rolling circle amplification (RCA), recombinase polymerase amplification (RPA), strand-displacement amplification (SDA), nucleic acid sequence-based amplification (NASBA), and exponential amplification reaction (EXPAR). The first mode may be a Cassie mode and the second mode may be a Wenzel mode. The first mode may be a slippery Wenzel mode and the second mode may be a sticky Wenzel mode.

In another example implementation, a sensor chip for detecting an analyte of interest may include but is not limited to a structure comprising a plurality of walls that define a plurality of air gaps in the structure, wherein each wall of the plurality of walls may include a plurality of surfaces. The sensor may include a functional layer, wherein the functional layer may be coated on the plurality of walls, wherein the functional layer comprises an extraction component to extract an analyte of interest, at least one amplification initiator to amplify the analyte of interest after extraction, and a material coating the plurality of walls providing an initial surface energy for at least a portion of the plurality of surfaces of the plurality of walls. The initial surface energy of at least the portion of the plurality of surfaces of the plurality of walls provided by the material coating may change when the analyte of interest is present.

One or more of the following example features may be included. The functional layer may further comprise a binding material to bind to the analyte of interest after amplification in a solution phase. The change in the initial surface energy due to the binding material binding amplicons in solution on at least the portion of the plurality of surfaces of the plurality of walls may prevent a fluid sample comprising the analyte of interest from exiting the plurality of air gaps. The change in the initial surface energy due to amplicons formed on at least the portion of the plurality of surfaces of the plurality of walls may prevent a fluid sample comprising the analyte of interest from exiting the plurality of air gaps after amplification in a solid phase. Multiple amplification initiators may be immobilized on at least the portion of the plurality of walls in spatially separated zones to enable multiplexing detection. A percentage of the initial surface energy change may quantitatively determine a concentration of the analyte of interest. Each amplification initiator may have a variable base, and a matching base that initiates an elongation of the each amplification initiator that produces the percentage of the initial surface energy change indicating a single nucleotide polymorphism (SNP) site. The plurality of walls coated with the functional layer may be fabricated from one of a micropillar array and a nanopillar array. Extraction and amplification may be conducted on the functional layer via at least one of a solution phase amplification and a solid phase amplification mechanism. The analyte of interest may be one of a DNA sequence, a RNA sequence, a single nucleotide polymorphism and multiple nucleotide polymorphism. The analyte of interest may be a complementary DNA (cDNA). An RNA target may form a heteroduplex with a trigger DNA to initiate amplification. The analyte of interest may be a microRNA sequence and a splinted ligation may extend a short microRNA sequence. The analyte of interest may be a microRNA sequence and a poly(A) tail may be added to a short microRNA sequence. The sensor may further include an additional component tagging one of an amplicon and a reverse primer to enhance the change of the initial surface energy on at least the portion of the plurality of surfaces of the plurality of walls. The analyte of interest may be amplified by one of loop-mediated isothermal amplification (LAMP), helicase dependent amplification (HDA), rolling circle amplification (RCA), recombinase polymerase amplification (RPA), strand-displacement amplification (SDA), nucleic acid sequence-based amplification (NASBA), and exponential amplification reaction (EXPAR).

The details of one or more example implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

### BRIEF DESCRIPTION OF THE DRAWINGS

In the drawings, like reference characters/symbols may generally refer to the same parts throughout the different views. For the purposes of clarity, not every component may be labeled in every drawing. Also, the drawings are not necessarily to scale, emphasis instead generally being placed upon illustrating the principles of the disclosure. In the following description, various embodiments of the present disclosure are described with reference to the following drawings, in which:

FIG. 1 shows an example sensor structure with integrated functional components for target extraction and binding in accordance with some embodiments of the present disclosure;

FIG. 2 shows an example solution phase amplification with the integrated sensor structure for extraction/binding/amplification/detection of target nucleic acid sequences, in accordance with some embodiments of the present disclosure;

FIG. 3 shows an example solid-phase amplification with the integrated sensor structure for extraction/binding/amplification/detection of target nucleic acid sequences, in accordance with some embodiments of the present disclosure;

FIG. 4 shows an example sensing mechanism of Cassie-to-Wenzel wetting mode transition, in accordance with some embodiments of the present disclosure;

FIG. 5 shows an example sensing mechanism of wetting mode transition from a slippery Wenzel mode to a sticky Wenzel mode, in accordance with some embodiments of the present disclosure;

FIG. 6 shows a schematic of an example method of detecting a RNA target using the wetting based micro-pillar array in the absence of reverse transcriptase in accordance with some embodiments of the present disclosure;

FIG. 7A and 7B show schematic of example methods of detecting a short RNA target using the wetting based micro-pillar array in accordance with some embodiments of the present disclosure;

FIG. 8 shows an example workflow of the sensor for extraction/amplification/detection steps, in accordance with some embodiments of the present disclosure;

FIG. 9 shows a schematic of an example multiplexing detection method for different nucleic acid targets using the wetting based micro-pillar array, in accordance with some embodiments of the present disclosure;

FIG. 10 shows a schematic of example methods to quantify the detection of nucleic acid targets using the wetting based micro-pillar array, in accordance with some embodiments of the present disclosure;

FIG. 11 shows a schematic of an example method of quantitative detection of nucleic acid targets using the wetting based micro-pillar array with a gradient coverage of the functional surface, in accordance with some embodiments of the present disclosure;

FIG. 12 shows a schematic of an example method of a SNP detection for a nucleic acid sequence using the wetting based micro-pillar array, in accordance with some embodiments of the present disclosure;

FIG. 13 shows a flow chart of an example method for producing an integrated molecular sensor, in accordance with some embodiments of the present disclosure;

FIG. 14 shows a schematic of an example method for producing a silicon-based integrated molecular sensor coated with functional layers, in accordance with some embodiments of the present disclosure;

FIG. 15 shows a schematic of an example method for producing a SU8-based integrated molecular sensor coated with functional layers, in accordance with some embodiments of the present disclosure;

FIG. 16 shows an example hole-like mold for forming pillar-like structure in a base substrate, in accordance with some embodiments of the present disclosure;

FIG. 17 shows a flow chart of an example method for manufacturing an integrated molecular sensor using an imprinting process, in accordance with some embodiments of the present disclosure;

FIG. 18 shows a schematic of an example method of conjugating an oligo, to a sensor surface via glutaraldehyde crosslinking, in accordance with some embodiments of the present disclosure;

FIG. 19 shows an example schematic for applying a hydrophobic layer to a sensor after the attachment of oligos, in accordance with some embodiments of the present disclosure;

FIG. 20 shows a schematic of an example method for attaching fluorinated oligos, to a fluorinated-silane sensor surface, in accordance with some embodiments of the present disclosure;

FIG. 21 shows a schematic of an example method for attaching a cholesterol tagged oligo, in accordance with some embodiments of the present disclosure;

FIG. 22 shows a schematic of an example method of conjugating an oligo, to a sensor surface via a carboxyl-to-amine crosslinking method, in accordance with some embodiments of the present disclosure; and

FIG. 23 shows a schematic of an example method of conjugating an oligo, to a sensor surface via copper catalyzed azide alkyne cycloaddition (CuAAC) click chemistry, in accordance with some embodiments of the present disclosure.

Like reference symbols in the various drawings may indicate like elements.

### DETAILED DESCRIPTION

In general, in various embodiments, the present disclosure relates to devices and methods for the detection of nucleic acid analytes in a fluid (e.g., liquid) sample. More specifically, the devices and methods integrate all necessary steps in conventional polymerase chain reaction (PCR) into one single sensor “chip” and may have a high affinity for an analyte, may be capable of detecting nucleic acid analytes in low concentrations with either solution phase amplification or solid-phase amplification, and may provide a (e.g., optical) signal as a notification to an end user upon detection. The devices and methods may also enable multiplexing and quantitative detection of DNA/RNA from a variety of viruses, bacteria, pathogen, fungi, etc. In the gene sequencing analysis, the devices and methods may also provide a rapid diagnostic for single nucleotide polymorphism (SNP) and multiple nucleotide polymorphism (MNP) as well.

To provide an overall understanding of the disclosure, certain example embodiments will now be described, including devices, methods of making the devices, and methods of detecting an analyte target molecule of interest (e.g., in a fluid sample or otherwise). However, the devices and methods described herein may be adapted and modified as appropriate for the application being addressed and the devices and methods described herein may be employed in other suitable applications. All such adaptations and modifications are to be considered within the scope of the disclosure.

Throughout the description, where compositions and devices such as a sensor are described as having, including, or comprising specific components, or where processes and methods are described as having, including, or comprising specific steps, it is contemplated that, additionally, there are compositions and devices of the present disclosure that consist essentially of, or consist of, the recited components, and that there are processes and methods according to the present disclosure that consist essentially of, or consist of, the recited processing steps. The terminology used herein is for the purpose of describing particular implementations only and is not intended to be limiting of the disclosure. As used herein, the singular forms “a”, “an” and “the” are intended to include the plural forms as well, unless the context clearly indicates otherwise. As used herein, the language “at least one of A and B” (and the like) as well as “at least one of A or B” (and the like) should be interpreted as covering only A, only B, or both A and B, unless the context clearly indicates otherwise. It will be further understood that the terms “comprises” and/or “comprising,” when used in this specification, specify the presence of stated features, integers, steps (not necessarily in a particular order), operations, elements, and/or components, but do not preclude the presence or addition of one or more other features, integers, steps (not necessarily in a particular order), operations, elements, components, and/or groups thereof

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a device or a method described herein can be combined in a variety of ways without departing from the spirit and scope of the present disclosure, whether explicit or implicit herein. For example, where reference is made to a particular feature, that feature can be used in various embodiments of the devices of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments can be variously combined or separated without departing from the present teachings and disclosure. For example, it will be appreciated that all features described and depicted herein can be applicable to all aspects of the disclosure described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

Where a percentage is provided with respect to an amount of a component or material in a composition such as a polymer, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remains operable. Moreover, two or more steps or actions can be conducted simultaneously.

At various places in the present specification, features are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or example language herein, for example, “such as” or “including,” is intended merely to better show the present disclosure and does not pose a limitation on the scope of the disclosure. No language in the specification should be construed as indicating any non-claimed element as essential to the practice of the present disclosure.

Various aspects of the disclosure are set forth herein under headings and/or in sections for clarity; however, it is understood that all aspects, embodiments, or features of the disclosure described in one particular section are not to be limited to that particular section but rather can apply to any aspect, embodiment, or feature of the present disclosure.

Nucleic acid based molecular tests may be the most accurate diagnostic assays; however, they typically require multiple steps, including sample preparation, amplification, and detection, most often requiring the use of additional instrumentation, including centrifuges, thermocyclers, heating sources, plate readers, etc. This adds to costs, complexity, and delays results. An ideal point-of-care (POC) medical diagnostic assay is low cost, simple, rapid, instrument-free, and capable of qualitative and/or quantitative nucleic acid detection. The two major categories of POC molecular tests include microfluidic based devices and paper-based tests, but each category has its own example and non-limiting disadvantages. For lab-on-a-chip based microfluidic sensors, the sample preparation and amplification steps are typically off-chip processes that include cell lysis, washing, centrifugation, filtration, and elution. Although some microfluidic devices have integrated sample preparation on chip, the device design is highly complicated with many different functional equipment components, such as fluid pumps, stop valves, mixing chambers, etc. to manipulate fluid and reagent movement, splitting, distribution, and mixing. The complicated design not only increases the manufacturing cost but also makes the device prone to fabrication defect and operation error. Other types of microfluidic sensors use plastics as the base materials for the microfluidic network; however, thermal cycles required by the extraction step (such as lysis) and amplification step, pose problems for the plastics used, as they have poor thermal properties that lead to defects and reliability concerns.

Another common test format is paper-based test, most often using lateral flow technology. Most paper-based molecular tests only use paper to conduct the lateral flow detection of amplicons or to conduct lysis of cells, but not as a substrate for amplification. The full integration of these steps on cellulose or nitrocellulose paper is still a great challenge. Furthermore, the opaque wicking fibers used in lateral flow detection often make the quantitative optical detection difficult. On the other hand, the real-time polymerase chain reaction (PCR), which is the current standard for the amplification step in a molecular test, requires heavy use of instruments (e.g., thermocycler, centrifuge, etc.), several hours of assay time, and multiple sample preparation steps performed by a well-trained lab technician. Thus, the present disclosure may alleviate some or all of these disadvantages by providing a simple and rapid molecular test (without using any instruments) that can fully integrate some or all three steps into a single chip. In some implementations, as will be discussed in greater detail below, the single chip may include a functional layer, which may include, e.g., an extraction component and an amplification initiator. In some implementations, the functional layer may include an extraction component, an amplification initiator, and a binding material.

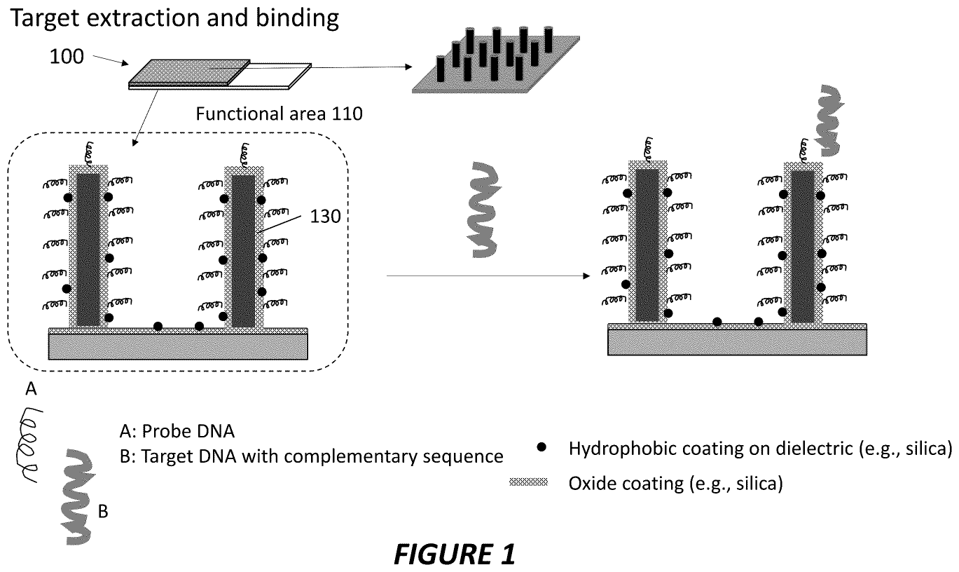

FIG. 1 shows a sensor 100 with an example micropillar structures as a functional sensing area 110 to detect target DNA/RNA sequences. The cylindrical micropillars are used only as non-limiting examples only rather than limitation. Other shapes and sizes of pillars or other geometrical structures may be used for the same purpose, for example, microspheres, nanospheres, microparticles, nanoparticles, square shaped cylinders, pyramid shapes, other reentrant structures, reversed cone arrays, hole arrays, micro-scaled islands and/or nano-scaled islands configured with any other shapes or irregular shapes and/or an aggregation of these structures and/or combination thereof. The structures in the functional area 110 may be made from a wide range of materials, for a non-limiting example, dielectric materials or metal including silica, titanium dioxide, hafnium oxide, polymer, plastic, PDMS, gold, silver, aluminum, copper, iron, and the like, and combinations thereof. Furthermore, these structures (e.g., microstructures and/or nanostructures) may be separated from each other to form a plurality of air gaps/voids. Air gaps and voids may be used interchangeably. The sizes are not limited to micrometer scales, and examples may range from 1 micrometer to 1000 micrometers, they can also be any smaller scale than 1 micrometer (e.g., nanometer scale as small as 0.1 nanometer) or larger scale than 1000 micrometers (e.g., millimeter scale as large as 10 millimeters). In some implementations, separation distances between (e.g., adjacent) microstructures and/or nanostructures may be as close as about 0.1 nanometers (“nm”). In some variations, the separation distance may be in the range from 0.1 nm to 10,000 microns. It has been discovered that in some implementations, the micropillar array acts not only as a signal transducer for the wetting mode-based sensing in the detection step but also works as a functional component to facilitate the lysis and extraction process when the appropriate size, shape, and interpillar spacing are selected. For a non-limiting example, the micropillar 110 may be a cylinder shape. Indeed, in addition to being cylindrical, the micropillar may be rectangular or of other shapes. Typical depths for micropillar may range between about one (1) nm and about one (1) centimeter, or between about 500 nm and about 1 millimeter, or between about 1 micrometer and about 100 micrometers. Typical diameters for cylindrical micropillars may range between about one (1) nm and about one (1) centimeter, or between about 500 nm and about 1 millimeter, or between about 1 micrometer and about 100 micrometers. Typical spacing between adjacent micropillars may range between about one (1) nm and about one (1) centimeter, or between about 500 nm and about 1 millimeter, or between about 1 micrometer and about 100 micrometers.

In some embodiments, cell lysis is achieved through the shear-force driven mechanical disruption of the cell membrane as a result from the fluid flow across the micropillar array or as a result from a hydrodynamic pressure applied to the micropillar array. In some implementations, mechanical disruption techniques may be used along with chemical disruption techniques such as the use of lysis buffers that will be described in the following text.

In some implementations, a thin layer (e.g., thickness from 0.1 nm to several hundred micrometers) of oxide coating 130 (e.g., silica, titanium dioxide, hafnium oxide, and the like, and combinations thereof) may be used for dual purpose as a color generator for colorimetric readout and as binding and extraction materials for nucleic acid target. The oxide coating layer 130 may be further treated with strong oxidizing agents (for example, hydrogen peroxide, potassium permanganate, alkali, ultraviolet radiation, TEMPO, etc.) to generate a carboxylated surface to facilitate nucleic acid capture and extraction.

Silica-coated material has a reversible binding affinity that is dependent on salt concentration. The nucleic acid (e.g., from DNA) binds under high-salt conditions, and is released under low-salt conditions. The nucleic acid and the silica each have a Debye double layer. The Debye double layer represents the distance that it takes for the electrostatic potential to decay, and it is a function of ionic concentration: the higher the salt concentration, the thinner the double-layer. Under high salt solutions, DNA and silica can move closer together and attractive Van der Waals forces overcome repulsive electrostatic forces (nucleic acids and silica both have negative surface charge). Also, lowering the pH decreases the negative charge density on the surface of the silica, which may reduce the repulsive force between a nucleic acid and silica. A nucleic acid also reversibly binds to the carboxylated surface in the presence or absence of polyethylene glycol (PEG) in varying salt concentrations.

In a similar mechanism to that of silica coatings, the concentration of PEG and salt is very important for nucleic acid extraction where the electrostatic potential (or repulsive force) must be overcome by attractive Van der Waals forces.

The oxide coating layer may be on a separate standalone zone on the sensor 100 from the functional sensing area 110 (i.e., micropillar area) on the same chip. In some implementations, a material (e.g., an additional hydrophobic material) coating is used along with micropillar structure 110 as the signal transducer to facilitate a target binding induced wettability change (or wetting state change) as a readout signal (wetting mode sensing will be described in the following sections).

In some implementations, binding materials, also referred to herein as receptors (for example, peptide, deoxyribonucleic acid (DNA), ribonucleic acid (RNA), peptide nucleic acid (PNA), oligonucleotides, molecularly-imprinted polymer (MIP) materials, aptamers, slow off-rate modified aptamers (SOMAmers), affirmers, antibodies, hormones, coordination complex, metal organic framework (MOF) materials, porous coordination polymer materials, and combination thereof) may be immobilized via physical adsorption or chemical bonding on the sensor surface to assist capturing the target DNA/RNA. In some implementations, receptors may consist of complementary sequences of target DNA/RNA either with part or the whole length of the target DNA/RNA sequence.

In some implementations, an extraction component may include an extraction lysis buffer (for a non-limiting example, NP-40 lysis buffer, chaotropic salt, sodium dodecyl sulfate lysis buffer, ammonium-chloride-potassium lysis buffer, and the like), a binding buffer (for a non-limiting example, chaotropic salt such as guanidine hydrochloride, urea, NaI, and the like or any molecules that can disrupt hydrogen bonding network between water molecules such as ethanol, n-butanol, phenol, isopropanol, chloroform, and the like) and/or an oxide coating layer (e.g., silica), where the extraction component may be used along with the silica coated chips to extract the target DNA/RNA from the sample matrix (e.g., whole blood, serum, saliva, etc.). High concentration of chaotropic salts facilitate disrupting the structure of macromolecules (e.g., proteins) and freeing the DNA/RNA from water to enable its binding to the coated oxide layer 130 (e.g., silica). In some other implementations, reagents other than chaotropic agents may be used to extract and eluent DNA/RNA from sample matrix. For example, amino acids, chitosan polymer, kosmotropic salts (e.g., ammonium sulfate), etc.

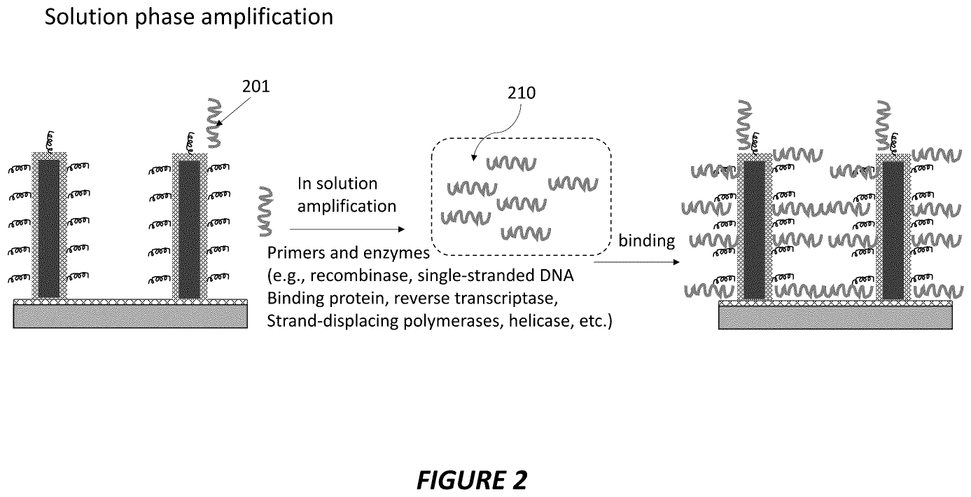

FIG. 2 shows an example method of amplification in solution phase. After extraction of target nucleic acids 201 from sample matrix, the sensor chip with the functional micropillar area as shown in FIG. 1 may be transferred to a tube (or other viable object) with amplification reagents in solution. The extracted target nucleic acids may be eluted (e.g., using hypotonic buffer) from the sensor chip into the solution or remain adsorbed on the sensor chip for subsequent amplification (e.g., isothermal amplification). In some implementations, such a solution may contain an amplification initiator (e.g., a primer such as an allele- specific primer, an oligonucleotide, an oligopeptide or other appropriate oligomer capable of being used with the present disclosure—that is long enough for specificity and short enough to “easily” bind to a template) or multiple primers to initiate amplification of the target sequence in solution phase and/or enzymes (such as recombinase, single-stranded DNA binding protein, reverse transcriptase, strand-displacing polymerases, helicase, etc.) to induce amplification of the target sequence in solution phase.

Amplification methods may include but are not limited to loop-mediated isothermal amplification (LAMP), helicase dependent amplification (HDA), rolling circle amplification (RCA), recombinase polymerase amplification (RPA), strand-displacement amplification (SDA), nucleic acid sequence-based amplification (NASBA), and exponential amplification reaction (EXPAR).

Amplification is carried out through either a thermocycling process or isothermal process in the presence of a solution of DNA/RNA target template, nucleotides (e.g., dNTP) and a thermostable polymerase (in the case of thermocycling) or polymerase along with other enzymes mentioned above (in the case of isothermal process).

In some implementations, amplification products 210 may be hybridizing with the probe DNA/RNA via Watson and Crick base pairing between A-T or G-C bases or may be binding to immobilized receptors via different types of molecular interactions (e.g., hydrogen bonding, van der Waals forces, hydrophobic interactions, and electrostatic forces). The hybridizing and/or binding of amplification products induce a surface energy change resulting in either a color change or a visible wetting mode changes as described in the above wetting mode-based sensing principle.

In some implementations, the amplification products may be quantified based on the percentage of color change on the sensor surface to achieve a quantitative detection of the target nucleic acids.

In yet another embodiment, the forward primer attached to the surface may serve as starting point for a solid-phase isothermal amplification (e.g., RPA).

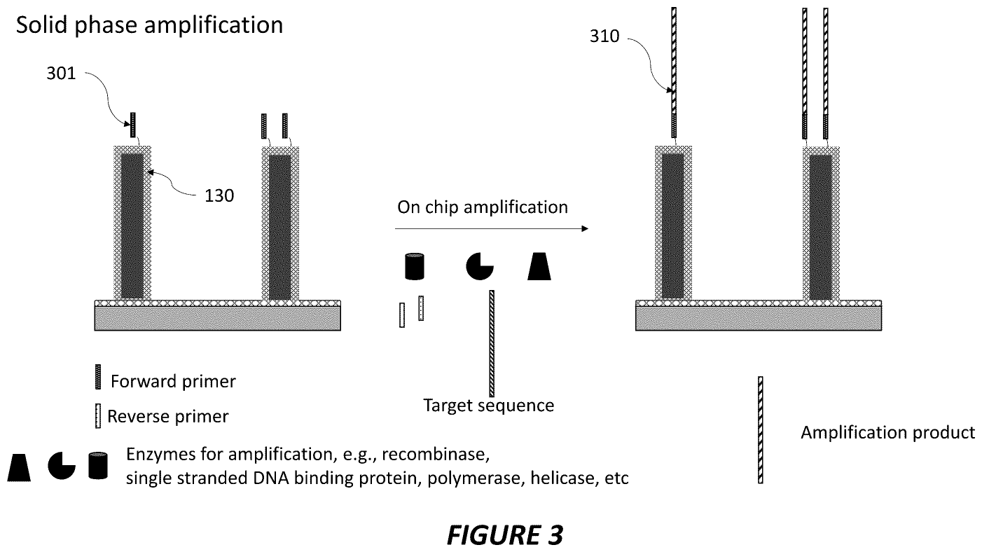

FIG. 3 shows a non-limiting example of solid phase amplification induced surface energy change. In some implementations, primers (for example, a forward primer) 301 may be immobilized on the sensor surface via either physical adsorption or chemical covalent bonding. Primers may be directly designed from PCR or HDA methods such as primer-BLAST and may also be modified with necessary attachment or spacers. Again, the disclosed sensor chips may be coated with an oxide layer 130 (e.g., silicon dioxide, titanium dioxide, hafnium oxide, and the like, and combinations thereof) as an extraction material along with the forward primers and chaotropic salt binding solution to extract target DNA/RNA from the sample matrix.

Some sample matrices may require additional treatment in traditional sample preparation. For example, blood plasma requires blood cell removal because hemoglobin can inhibit polymerase activity. However, the disclosed fully integrated sensor chip can easily separate the blood cell from the polymerase to eliminate the need of a centrifuge because these two components are in two separate steps (blood cells are physically separated from the analyte target extracted with the sensor chip in the extraction step while polymerase are added in the amplification step).

Sensor chips may be further exposed to a solution that contains other primers (for example, a reverse primer) and enzymes (such as recombinase, single-stranded DNA binding protein, reverse transcriptase, strand-displacing polymerases, helicase, etc.) to induce an on-chip solid phase amplification of the target sequence when the target is present.

The elongation of the primer with amplicon 310 on the sensor surface leads to a surface energy change that may be detected as a color change or visually detected by a wetting mode change. In some implementations, the above-noted binding material (e.g., aptamer, antibody, MIP, etc.) used to bind the analyte of interest after amplification may be used in the solution phase, but need not be used in solid phase amplification due to the elongation of the primer/amplification initiator (e.g., amplicon) being sufficient enough to change the surface energy creating the wetting mode change.

In some implementations, both forward primer and reverse primer may be immobilized on the sensor surface via either physical adsorption or chemical covalent bonding. Bridge amplification may be induced when analyte targets are present. In bridge amplification, the analyte target may first bind to forward primers and the elongation of forward primers produces a tethered reverse strand complementary to the analyte target. Then the reverse strand may denature and bend over towards the solid surface and binds to another reverse primer immobilized on the solid surface to form a bridge connection. The elongation of reverse primers produces a surface tethered analyte target strand. Both forward and reverse strands produced from bridge amplification may induce a surface energy change that may be detected as a color change or visually detected by a wetting mode change.

In some other implementations, the signal may be weak, and it may take a long time to accumulate more amplified product to generate a strong signal. In this case, the amplicon or the reverse primer may be tagged with one or more than one hydrophilic component (e.g., a protein, peptide, etc.) to further enhance the signal. Solid-phase amplification has the example and non-limiting advantage that it is highly adaptable to multiplexed amplification, particularly when detection is facilitated by the surface on which amplification takes place. The functional sensing surface (areas, zones, etc.) may be functionalized with different primers for multiplex detection.

No matter if it is solution phase or solid phase amplification, the final readout or color change is induced by the wetting mode change resulting from the introduction of the analyte. There are two different example wetting mode changes that are energetically favorable: from a Cassie mode to Wenzel mode transition, and from a slippery Wenzel mode to a sticky Wenzel mode. Both mode changes will be described in the following sections. There may be other example wetting mode changes that are not energetically favorable, for example, from Wenzel mode to Cassie mode transition, and from a sticky Wenzel mode to a slippery Wenzel mode. The following section describes the energetically favorable transitions only for the purpose of illustration and not limitation.

Two wetting modes can exist when a fluid (e.g., liquid) interacts with a hydrophobic solid surface. Cassie mode is known to prevent fluid (e.g., liquid) from penetrating into air gaps, such as the air gaps between the above-noted pillars, by forming a solid-liquid-air interface, so that the liquid is extremely mobile (i.e., “non-sticky”) and can roll freely on the solid surface using only gravity when the solid surface is tipped, tilted or flipped. Wenzel mode is another wetting mode in which the fluid (e.g., liquid) penetrates into the air gaps of a structured surface, such as the air gaps between the above-noted pillars, which enables the fluid (e.g., liquid) to fully wet the surface of the structure and become immobile (i.e., “sticky”) on the solid surface. In the following text, “non-sticky” may be used as an interchangeable term with “slippery” to indicate a slippery state describing the mobile nature of a liquid on a designed surface when the surface is tilted or flipped, while “sticky” may be used interchangeably with “wet” to indicate a wet state of the liquid in which the liquid adheres to or is adsorbed by the solid surface, preventing the liquid from running off of the solid surface even when the surface is tilted or flipped. The physical origin of the “sticky” Wenzel mode is liquid pinning, which results from the interaction of liquid with the solid surface.

The in-flow and/or presence of a specific fluid, e.g., a fluid containing a target analyte of interest, in the air gaps/voids of a three-dimensional (“3D”) structure (such as the above-noted pillars) may be used in diagnostic devices, e.g., photonic crystals, micropillar arrays, multilayer dielectric stack, etc., and other colorimetric sensors, to verify or confirm the presence of the specific fluid. Indeed, and more particularly, a color change detectable in the visible spectrum of light refracted by the structure due to, inter alia, a difference in the refractive index of the structure because of the presence of the fluid in the air gaps/voids may be used to identify the nature of the fluid captured in the air gaps of the structure. In this type of sensor device, the sensing mechanism is based on a Cassie-to-Wenzel transition. More specifically, the initial wetting state may be a Cassie mode that prevents liquid from infiltrating into air gaps/voids, while the end wetting state upon analyte binding may be a Wenzel mode that allows liquid to infiltrate the air gaps/voids and adhere to or be adsorbed by the surface due to the changing surface energy caused by the binding.

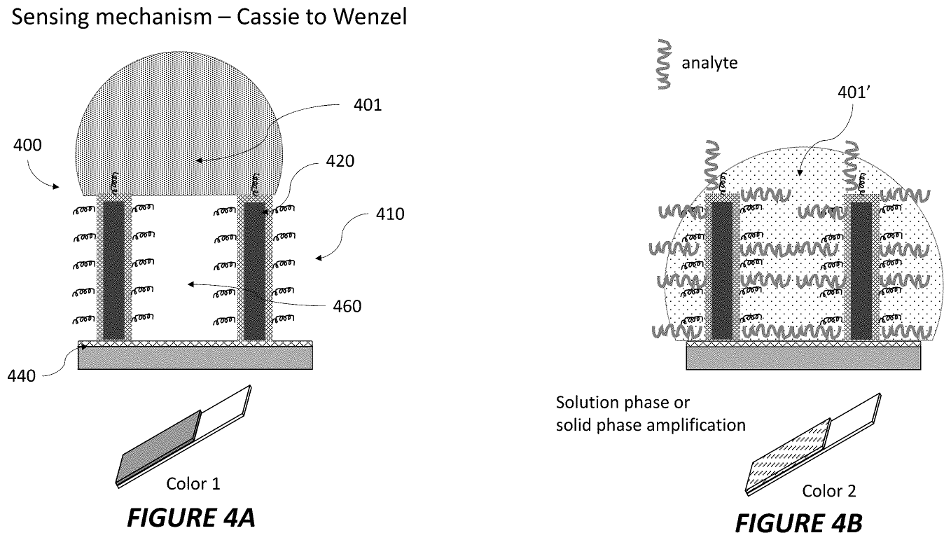

As shown in FIG. 4A, in some implementations, the three-dimensional (“3D”) sensor or sensing device 400 may include a transducer 410 (e.g., a micropillar array) and an indicator 440 (e.g., a colorimetric indicator 440) that are physically spaced from one another. In some applications, the transducer 410 may include a plurality of microstructures and/or nanostructures 420 (e.g., pillars, micropillars, nanopillars, and the like) having air voids and/or gaps 460 between adjacent microstructures and/or nanostructures 420.

The transducer 410 and indicator 440 may be structured and arranged to verify or confirm the presence (or absence) of a specific fluid (e.g., a fluid containing the target analyte of interest).

With reference to FIG. 4A, when the spacing between the micropillars are narrow enough, and because the sensor surface is hydrophobic, sensor chip may be initially in a low surface energy state (i.e., poor wetting or hydrophobic). A fluid 401 that does not include the analyte of interest is not able to penetrate through the grooves 460 (i.e., a Cassie-Baxter wetting mode may be observed). In this case, a color 1 (e.g., blue) may be observed as an indication of the absence of analyte. In contrast, after exposing to analyte sequence and solution phase amplification, the amplification produces a sufficient amount of amplicons in solution and the binding of amplicon to receptors may cause a change in the surface energy sufficient enough to allow wetting mode transition from Cassie mode to Wenzel mode. For example, the sensor surface may be in a high surface energy state (i.e., good wetting or hydrophilic) due to the hybridization of the amplification product with the receptors (e.g., complementary probe DNA/RNA) on the sensor surface. As such, a full fluid penetration or partial penetration into the grooves 460 is triggered (i.e., a Wenzel wetting mode may be observed). The wetting mode change may be visualized as a wetting surface in contrast to a dry surface in the Cassie wetting mode and/or as an observed spectra change (reflection spectra, scattering spectra, or transmission spectra).

As a non-limiting example, the wetting mode change may also be visualized by integrating a structural color based thin film element (e.g., an oxide layer as shown in FIG. 1) to induce a color change from the original color (designated as “Color 1”) to a different color (designated as “Color 2”, e.g., red) upon fluid contact with such a thin film indicator element. It will be appreciated that other examples of visualization may be used without departing from the scope of the present disclosure. For instance, the presence of the liquid after tilting the sensor may also provide sufficient visualization.

Introduction of such an analyte of interest may induce an amplification reaction in the liquid phase (i.e., in solution) to generate a sufficiently high concentration of amplified product that will bind to the receptors on the sensor surface, or the introduction of such an analyte of interest may induce a solid phase amplification reaction on the solid surface of the sensor to generate a sufficiently high concentration of amplified product that is elongated from the primer immobilized on the sensor surface.

In some implementation, reversibility to Cassie mode after Cassie-to-Wenzel transition may also be used as an indication of the presence of analyte of interest. When the analyte of interest is not present, the fluid is not able to penetrate the grooves. When mixing the fluid containing no analyte of interest with lower surface tension liquid or surfactant molecules, Cassie-to-Wenzel transition is initiated, and fluid is able to penetrate the grooves. After removing the fluid from the sensor surface, the wetting mode is able to be reversed to Cassie mode, which indicates the absence of analyte. When the fluid containing the analyte of interest is mixed with lower surface tension liquid or surfactant molecules, Cassie-to-Wenzel transition is initiated, and fluid is able to penetrate the grooves. After removing the fluid from the sensor surface, the wetting mode is not able to be reversed to Cassie mode indicating the presence of analyte.

In some other implementations, it is possible to design a sensor that has a transition from a “non-sticky” Wenzel mode (i.e., a mode in which liquid is able to penetrate into the air gaps/voids of the 3D structure but the structure remains non-sticky) to a “sticky” Wenzel mode. In such implementations, the initial “non-sticky” Wenzel wetting state allows all liquids to penetrate the air gaps/voids; however, a liquid lacking an analyte of interest can roll off or be poured off the structure, reverting the surface of the structure to a dry state with no liquid in the air gaps/voids. In short, the liquid lacking the analyte of interest is incapable of sticking to the structure; hence, “non-sticky.” In the presence of a liquid containing a target analyte of interest, however, the end wetting state is a “sticky” Wenzel mode in which the liquid containing the target analyte of interest is retained in the air gaps/voids after removal of the sensor from the liquid sample.

According to some embodiments of the present disclosure, the in-flow and/or presence of a specific fluid, such as a fluid containing a target analyte of interest (e.g., fuel containing contaminants, blood or saliva containing certain biomarkers for certain diseases or injuries, liquid containing certain drugs, and the like), may be detected using wetting-based colorimetric devices having colorimetric (e.g., dye, pigment, photonic crystal, and the like) indicators that are structured and arranged to exhibit an observable change in color upon contact with the fluid and the fluid adhering to, attaching to, and/or being adsorbed by the surface of the structure.

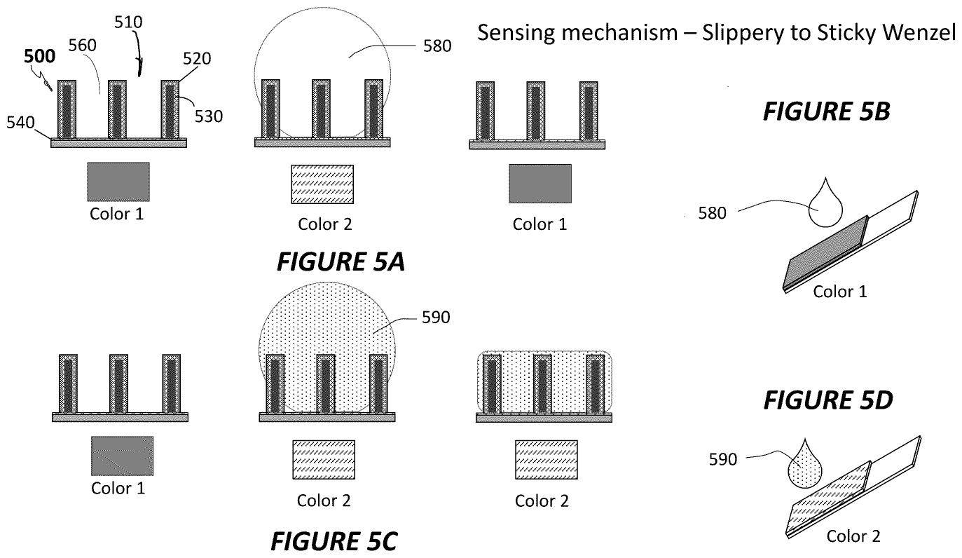

Referring to FIGS. 5A-5D, an example embodiment of a wetting-based colorimetric sensing device, or sensor, 500 is shown. In particular, sensor 500 is one that, as further described below, transitions from a “non-sticky” Wenzel mode to a “sticky” Wenzel mode when an analyte of interest is present in a fluid to induce an amplification either in solution phase or on chip surface.

As shown in FIG. 5A, in some implementations, the three-dimensional (“3D”) sensor or sensing device 500 may include a transducer 510 (e.g., a micropillar array) and an indicator 540 (e.g., a colorimetric indicator 540) that are physically spaced from one another. In some applications, the transducer 510 may include a plurality of microstructures and/or nanostructures 520 (e.g., pillars, micropillars, nanopillars, and the like or combinations thereof) having air voids and/or gaps 560 between adjacent microstructures and/or nanostructures 520.

In some implementations, the (e.g., 3D) transducer 510 may include a roughened surface comprising microscale and/or nanoscale features. The microscale and nanoscale features may include microstructures or nanostructures 520 (e.g., a plurality of pillars, micropillars, or nanopillars) or combination of both. In some applications, the microstructures and/or nanostructures 520 (e.g., pillars, micropillars, and/or nanopillars) may be manufactured of a dielectric or insulative material (e.g., silica, titanium dioxide, silicon nitride, polymer, PDMS, and the like), a (e.g., organic, inorganic, or hybrid) molecularly-imprinted polymer (MIP) material, a metallic material (e.g., gold, silver, aluminum, and the like), a metallic material coated with a MIP material, and combinations thereof

In some embodiments, the surface of the microstructures and/or nanostructures 520 (e.g., pillars, micropillars, and/or nanopillars) may be coated with a (e.g., thin) layer of a binding material 530 (e.g., analyte receptor), such as MIP material, aptamer material, slow off-rate modified aptamers (SOMAmers), affirmers, antibodies, hormones, peptide, deoxyribonucleic acid (DNA), ribonucleic acid (RNA), peptide nucleic acid (PNA), oligonucleotides, coordination complex, metal organic framework (MOF) materials, porous coordination polymer materials, and so forth, or combinations thereof

In some implementations, the thickness of the binding material (e.g., analyte receptor) coating 530 may range from about 1 Angstrom (e.g., in the case of a molecular monolayer) to about a thickness of approximately the distance between the adjacent microstructures and/or nanostructures 520 (e.g., the plurality of pillars, micropillars, and/or nanopillars). The binding materials along with coating materials (e.g., silica) may be used as the solid phase extraction layer to facilitate the extraction of nucleic acids from complex sample matrix (e.g., blood, CSF, saliva, urine, tears, nasal fluid, fecal matter, etc.).

Advantageously, the binding material (e.g., analyte receptor) coating 530 may also include a binding material produced from hydrophobic components, so that the binding material is hydrophobic, or produced from hydrophilic binding materials coated with an additional hydrophobic layer, or combinations thereof. In some implementations, the binding material (e.g., analyte receptor) coating 530 may also include a specific binding enhancement layer or an additional layer to reduce non-specific binding from non-target substances in the liquid. Non-limiting examples of such a layer include polymer brushes, zwitterionic polymers, protein blocker agents and so forth.

The transducer 510 and indicator 540 may be structured and arranged to verify or confirm the presence (or absence) of a specific fluid (e.g., a fluid containing the target analyte of interest). For example, as shown in FIG. 5A, when a fluid 580 that does not contain the target analyte of interest contacts the surface of the sensor 500, the fluid 580 initially penetrates the air gaps/voids 560 disposed between the plurality of microstructures and/or nanostructures 520 in the transducer 510. The presence of the fluid 580 in the air gaps/voids 560 modifies the refractive index of the sensor 500, thus leading to an observable color change. In particular, the penetration of the fluid 580 into the air gaps/voids 560 when the sensor 500 is exposed to the fluid 580 may, initially, cause the sensor 500 to exhibit a different color (designated “Color 2” in FIG. 5A) from the original color (designated as “Color 1” in FIG. 5A) exhibited by the sensor 500 in air or ambient conditions (i.e., when the fluid 580 is absent).

Since, however, the fluid 580 does not contain the target analyte of interest, sensor 500 remains in a “non-sticky” Wenzel wetting mode. As a result, the fluid 580 can roll off the surface of the sensor 500 when the sensor 500 is tilted or flipped. The absence (e.g., runoff) of the fluid 580 returns the sensor 500 to its original color (i.e., “Color 1”), thereby providing indicia of an absence of the target analyte of interest in the fluid 580. FIG. 5B shows this phenomena schematically—i.e., tilting or flipping the sensor 500 causes the fluid 580 that does not contain a target analyte of interest to roll off the sensor 500, thereby causing the sensor 500 to exhibit its original color (i.e., “Color 1”).

As shown in FIG. 5C, when a fluid 590 that contains the target analyte of interest contacts the surface of the sensor 500, it too initially penetrates the air gaps/voids 560 between the microstructures and/or nanostructures 520 of the transducer 510. The target analytes of interest present in the fluid 590 induce either an amplification in solution or a solid-phase amplification on the sensor surface. In the solution phase case, the amplicons proceed to bind with the binding material 530 (e.g., the analyte receptor). In the case of solid-phase amplification, the surface energy of the solid surface (e.g., the microstructures and/or nanostructures 520 of the transducer 510) is modified due to the elongation of the primer. Both the binding of the target analytes of interest to the binding material 530 and the elongation of primers immobilized on the surface lead to a change in the surface energy within the surficial walls of the microstructures and/or nanostructures 520, a wetting of those surficial walls, and a transition of the sensor 500 to a “sticky” Wenzel state. As used herein, the term “surficial wall” means any surface of the wall. As before, the presence of the fluid 590 in the air gaps/voids 560 modifies the refractive index of the sensor 500, thus leading to an observable color change. In particular, the penetration of the fluid 590 into the air gaps/voids 560 when the sensor 500 is exposed to the fluid 590 may cause the sensor 500 to exhibit a different color (designated “Color 2” in FIG. 5C) from the original color (designated as “Color 1” in FIG. 5C) exhibited by the sensor 500 in air or ambient conditions (i.e., when the fluid 590 is absent).

The “sticky” Wenzel state of the sensor 500 created by either the binding of the target analytes of interest in the fluid 590 to the binding material 530 (e.g., the analyte receptor) or the elongation of primers on the solid surface prevent the fluid 590 from rolling off the surface of the sensor 500 when the sensor 500 is tilted or flipped (e.g., the fluid 590 remains trapped within the air gaps/voids 560 between the microstructures and/or nanostructures 520 of the transducer 510). Accordingly, the sensor 500 maintains the different color (designated “Color2” in FIG. 5C) and does not return to its original color (designated “Color1” in FIG. 5C) even when the sensor 500 is tilted or flipped, thereby providing indicia of the presence of the target analytes of interest in the fluid 590. FIG. 5D shows this phenomena schematically—i.e., tilting or flipping the sensor 500 does not cause the fluid 590 that contains the target analytes of interest to roll off the sensor 500, thereby causing the sensor 500 to exhibit a color (i.e. “Color2”) different from its original color (i.e., “Color1”).

Although FIGS. 5A-5D show a pillar-based micro- and/or nanostructure sensor 500 having three microstructures and/or nanostructures 520, that is done for the purpose of illustration rather than limitation. Indeed, any number of microstructures and/or nanostructures 520 (e.g., micro- and/or nanopillars), any shape of microstructures and/or nanostructures 520, and any arrangement or distribution of microstructures and/or nanostructures 520 on the surface of the indicator 540 may be used. For example, the microstructures and/or nanostructures 520 may be micro- and/or nanopillars formed in a periodic, aperiodic, and/or random array. The microstructures and/or nanostructures 520 may be corn-shaped to facilitate cell lysis. Furthermore, the microstructures and/or nanostructures 520 may be separated from each other to form a single air gap/void or a plurality of air gaps/voids 560.

Separation distances between (e.g., adjacent) microstructures and/or nanostructures 520 may be as close as about 0.1 nanometers (“nm”). In some variations, the separation distance may be in the range from 0.1 nm to 10,000 microns. In another example embodiment, rather than micro- and/or nanopillars 520, the transducer 510 may include, for the purpose of illustration rather than limitation: microspheres, nanospheres, microparticles, nanoparticles, micro-scaled islands and/or nano-scaled islands configured with any other shapes or irregular shapes and/or an aggregation of these structures and/or combination thereof. Furthermore, these microstructures and/or nanostructures 520 may be separated from each other to form a plurality of air gaps/voids 560.

In some implementations, the width (e.g., rectangular shaped) or the diameter (e.g., circular shaped) and the height of the micropillars and/or nanopillars 520 may range from about 0.001 nm to about 10,000 microns. Those skilled in the art can appreciate that selection of the size (e.g., diameter or other dimension) of the micropillars and/or nanopillars 520 may depend on the target analyte of interest; hence, the size of the micropillars and/or nanopillars 520 may be smaller than 0.001 nm or larger than 10,000 microns. Moreover, although the (e.g., circular) micropillar and/or nanopillar diameters and/or (e.g., rectangular) micropillar and/or nanopillar widths shown in FIGS. 5A-5D appear to be uniform in dimension, that, too, is done for the purpose of illustration rather than limitation. Indeed, in some variations, a variety of micropillar and/or nanopillar diameters or other dimensions may be formed on a single sensing device 500. As one non-limiting example, a hierarchical structure of microstructures and/or nanostructures 520 with two or more size dimensions may be formed.

In some other non-limiting examples, the surface of the sensor 500 may be a roughened surface that comprises random vertical deviations of a roughness profile from a mean line. The vertical deviation may range from about 0.001 nm to about 10,000 microns. Those of ordinary skill in the art can appreciate that, in some applications, the vertical deviation may be smaller than 0.001 nm or larger than 10,000 microns. When the surface roughness is below the visible optical wavelength (i.e., below 380 nm), the surface may visually appear to be flat to human eyes.

In some implementations, to prevent or limit fluids 580 that do not contain a target analyte of interest from becoming trapped within the air gaps/voids 560 between the microstructures and/or nanostructures 520 of the transducer 510, a hydrophobic material (e.g., Teflon, silane, and the like) may be coated on the surficial walls of the microstructures and/or nanostructures 520. Advantageously, the hydrophobic coating may be designed to repel all fluids 580 that do not contain the target analyte of interest, thereby allowing the fluid 580 to roll off the surface of the sensing device 500 and preventing the fluid 580 from becoming trapped within the air gaps/voids 560 between the microstructures and/or nanostructures 520. Those of ordinary skill in the art can appreciate that, in some applications, the microstructures and/or nanostructures 520 may be coated with a hydrophobic material as well as with the binding material 530 (e.g., an analyte receptor).

In some embodiments, the colorimetric indicator 540 is structured and arranged to exhibit a color change when a fluid is in contact with and/or sticking to (e.g., attached to, adhered to, adsorbed by, and the like) the surface of the colorimetric indicator 540. In some embodiments, the colorimetric indicator 540 is a dye, pigment, light emitting molecule and device, and combination thereof. In some embodiments, the colorimetric indicator 540 is a structural color indicator. Example structural color indicators include Bragg reflective coatings, photonic crystals, and interference-based thin film reflectors, multilayer dielectric stack, plasmonic crystals, plasmonic thin films, dielectric nanoparticles and nanostructures, metal nanoparticles and nanostructures, and the like, and combination thereof. The detectable visible color difference (i.e., the change of color) that results from liquid interaction with a structural color indicator is generally referred to as a structural color change. As one non-limiting example, there may be a color change in the visible spectrum of light refracted by the colorimetric indicator 540 once the fluid is introduced into (e.g., photonic crystals associated with) the colorimetric indicator 540. In other words, the introduction of the fluid may lead to a difference in the refractive index of the colorimetric indicator 540. Advantageously, the difference in the refractive index may be used to identify the nature of the fluid trapped within the air gaps/voids 560.

Another suitable structural color indicator may be, for example, a thin film of dielectric/metallic/semiconductor material deposited on another dielectric/metallic/semiconductor material (e.g., a silicon oxide thin film deposited on silicon, gold, or germanium deposited on silicon). It will be appreciated that any suitable structural color indicator may be used throughout without departing from the scope of the present disclosure.

In some implementations, the sensing device 500 may be used without the colorimetric indicator 540. For example, a fluid sticking to the surface of the sensing device 500 may be visualized upon the binding of target analytes of interest with the binding material 530 (e.g., an analyte receptor). A fluid that cannot roll off the surface of the sensing device 500 may be directly visualized by the naked human eye. Furthermore, the sensing device 500 may also be used without the microstructures and/or nanostructures 520 (e.g., pillars, micropillars, nanopillars, and the like) or colorimetric indicator 540. For example, a fluid sticking to a flat and smooth surface may be visualized upon the binding of a target analyte of interest with the binding material 530 (e.g., an analyte receptor) of the sensing device 500. More specifically, a fluid that cannot roll off the surface of the sensing device 500 may be directly visualized by the naked human eye. Therefore, any changes (e.g., a fluid sticking to the surface) as a result of binding of the target analytes of interest with the binding material may be considered as an indication of the presence of the target analyte, regardless of whether there is a colorimetric change.

In some implementations, the target of interest may be a RNA molecule, so a reverse transcription (RT) may be needed to first convert RNA into complementary DNA (cDNA) prior to the amplification. RT protocol may be performed with a reverse transcriptase kit containing primers, dNTP, a reverse transcriptase, e.g., Avian Myeloblastosis Virus (AMV) Reverse Transcriptase or Moloney Murine Leukemia Virus (M-MuLV, MMLV) Reverse Transcriptase, etc. The synthesized cDNA may be directly used for subsequent amplification and detection.

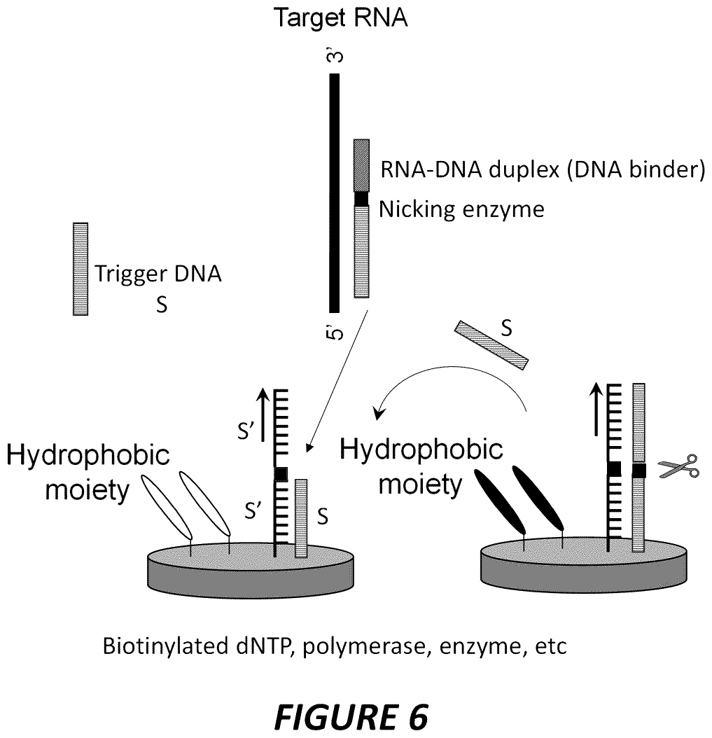

In some other implementations, RNA may be amplified without using a reverse transcriptase. FIG. 6 shows a non-limiting example of exponential amplification reaction (EXPAR) without reverse transcriptase. The template (S′) that is complementary to the trigger DNA (S) may be immobilized on the sensor surface along with an identical template sequence (S′). A binder DNA containing the trigger DNA (S) may be used to form a heteroduplex with target RNA. A restriction enzyme (e.g., BstNI) may be used as a nicking enzyme by selectively cleaving DNA within the RNA:DNA heteroduplex and release the trigger DNA to initiate EXPAR reaction. The signal may be generated either in solution phase from the trigger DNA or in solid-phase from the elongated templates with biotin labels, which may induce a further signal enhancement in the presence of a buffer with streptavidin.

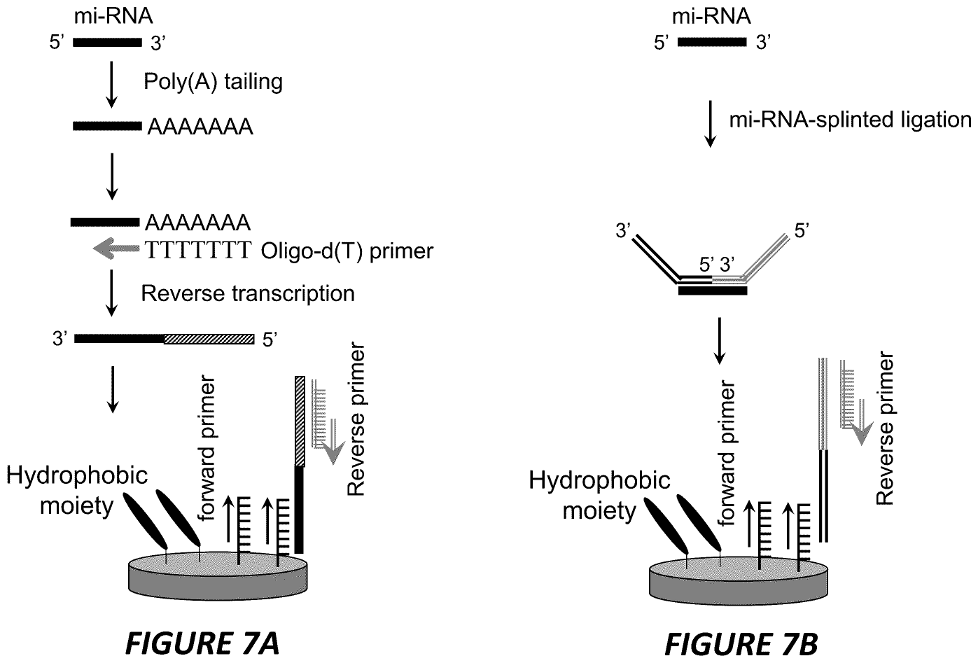

In some other implementation, the target RNA may have a short sequence (e.g., microRNA) that is about the same size as a conventional primer, which may make it difficult for the subsequent amplification step. To enable the detection of a short RNA sequence, a poly(A) tail may be added to the short RNA target and an Oligo-d(T) primer may be used for reverse transcription as shown in FIG. 7A. The miRNA-specific forward primer immobilized on the micropillar array will initiate the solid-phase isothermal amplification in the presence of the cDNA template (generated from RT reaction), a universal reverse primer, and suitable enzymes (e.g., polymerase) in the solution phase. The elongation of forward primers will result in a surface energy change, thus leading to a visible color change.

FIG. 7B shows another non-limiting example for detection of short RNA sequence with a non-RT based method by using splinted ligation to “lengthen” or extend the short miRNA sequence with additional probe sequences (i.e., additional DNA sequences extended beyond the short RNA sequence). A solid-phase isothermal amplification may be initiated by the immobilized specific forward (or reverse) primer along with other components, including a specific reverse (or forward) primer, an elongated template, and necessary enzymes in solution.

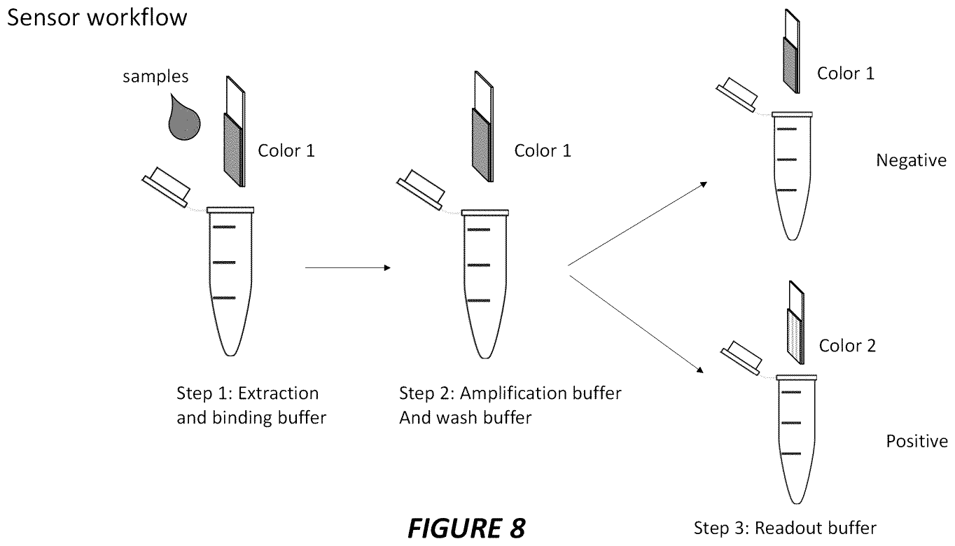

A non-limiting example of the sensor workflow is shown in FIG. 8. The sensor may be used as a smart material to detect target nucleic acid sequence without using any instruments for, e.g., low resource settings or at-home use. Examples of instruments include any electrical equipment and workstation such as thermocycler, heating bath, centrifuge, plate reader, battery powered equipment, etc.

The first step (e.g., Step 1 in FIG. 8)) may be extraction of the target DNA/RNA from the sample matrix (e.g., whole blood, serum, CSF, saliva, etc.) in the extraction and binding buffers. These buffers may include extraction lysis buffer (for a non-limiting example, NP-40 lysis buffer, sodium dodecyl sulfate lysis buffer, ammonium-chloride-potassium lysis buffer, and the like) and binding buffer (for a non-limiting example, chaotropic salt such as guanidine hydrochloride, urea, NaI, and the like or any molecules that can disrupt hydrogen bonding network between water molecules such as ethanol, n-butanol, phenol, isopropanol, chloroform and the like). The binding buffer may contain other reagents (for example, blocking reagent) to reduce the non-specific binding from unwanted proteins or enzymes in the sample matrix. After the extraction step, the target DNA/RNA may be bound to the sensor chip or may be eluted from the sensor chip using elution buffer (e.g., a low salt buffer) or distilled water.

In some implementations, RNA may need to be converted to cDNA sequence via a reverse transcription process. In some other implementation, a reverse-transcription free process may be used along with the subsequent amplification process. For example, an EXPAR reaction without reverse transcriptases. In some implementations, RNA target may be forming a heteroduplex with cDNA and a strand displacement DNA (dDNA) may be used to hybridize with the RNA target and release the cDNA for subsequent amplification.

The second step of the sensor workflow (e.g., Step 2 in FIG. 8) may be an amplification of the target (if present) either in solution phase or in solid phase on the sensor surface. The sensor chip may be inserted into a buffer solution containing components used for isothermal amplification (e.g., primers, polymerase enzymes, recombinase, etc.). Amplification methods may include but are not limited to loop-mediated isothermal amplification (LAMP), helicase dependent amplification (HDA), rolling circle amplification (RCA), recombinase polymerase amplification (RPA), strand-displacement amplification (SDA), nucleic acid sequence-based amplification (NASBA), and exponential amplification reaction (EXPAR). Washing may be also performed in this step with suitable washing buffers (for example, ionic surfactant SDS; non-ionic surfactant Tween 20, triton X-100; and zwitterionic surfactant CHAPS, etc.) to reduce the non-specific binding from non-target proteins and enzymes. In the case of solution phase amplification, the amplicons are produced in solution and may be hybridized with the probe immobilized on the sensor chip. In the solid-phase amplification, the primer or template immobilized on the sensor surface will be elongated to generate the signal. In some implementations, the amplification time may be in the range from 1 second to 24 hours.

The third step of the sensor workflow (e.g., Step 3 in FIG. 8) may be a signal readout using another buffer (e.g., PBS buffer). When there is no target analyte of interest present, the sensor chip may be either dry or exhibit its original color (i.e., “Color 1”). When there is a target analyte of interest present, the chip may be either wet or exhibit a color (i.e., “Color 2”) different from its original color (i.e., “Color 1”). In some implementations, the sensor chip may be dipped into other buffer solution containing an enhancement agent to further enhance the signal readout. For example, dipping into streptavidin buffer when the primer is pre-labelled with biotin.

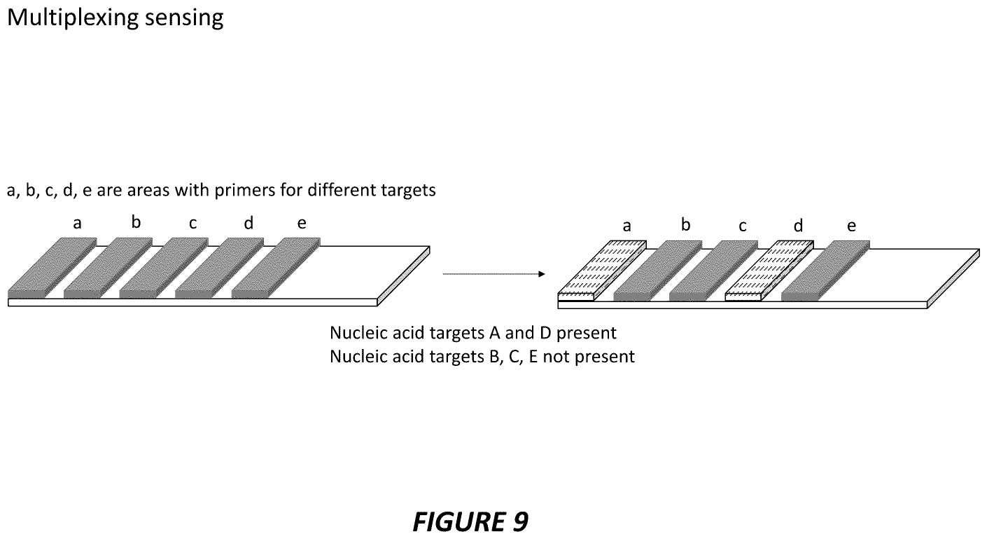

In some implementations, FIG. 9 demonstrates a non-limiting example of multiplexed sensor with immobilized primers for different nucleic acid targets as 5 representative sensing zones (areas) a, b, c, d, and e. The spatial separation of the target eliminates the need to use different labels to differentiate each target. Different immobilization techniques may be used, for a non-limiting example, a drop casting method may be used to physically attach the primers to the sensor surface at different locations. Alternatively, the drop casting method may be used along with a click chemistry reaction to form chemical bonding between the primers and different target-specific areas across the sensor surface. For example, primers may be modified with a 5′ hexynyl group that reacts with an azido functionalized sensor surface via a Copper-catalyzed azide-alkyne cycloaddition (CuAAC) click chemistry reaction. In some implementation, primers may be immobilized via EDC-NHS Carbodiimide cross-linking. When there is a target present (e.g., targets A and D), the corresponding functional area (e.g., area a and d) exhibits a different color (and/or a sticky surface). When there is no target present (e.g., targets B, C, and E), the corresponding functional zone (area) (e.g., area b, c, and e) remains its original color and does not exhibit a different color (and/or remains unsticky).

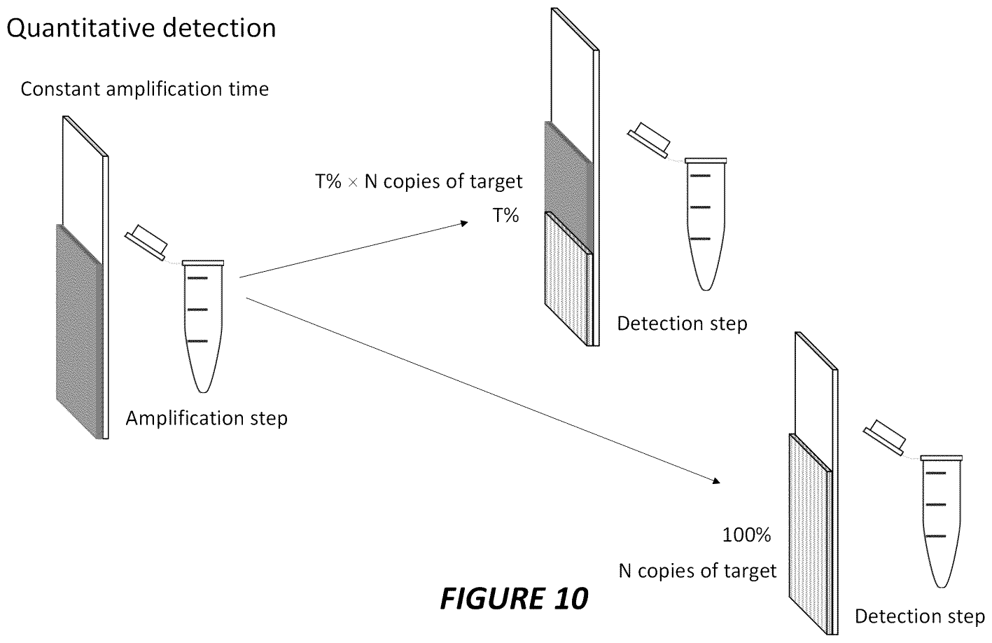

FIG. 10 shows a non-limiting example of quantitative detection of nucleic acid. There are two example ways to quantify the target nucleic acid concentration assuming the surface coverage of primers is homogeneous. The first way is to monitor the percentage of the color change (or wetting mode change) in the detection step while keeping the amplification time constant in the amplification step. For example, if the 100% color change corresponds to a target nucleic acid concentration of N copies, then T % color change (or wetting mode change) indicates a target nucleic acid concentration of T %×N copies. The second way is to monitor the amplification time that is required to reach a certain percentage of color change (or wetting mode change), for example, a 100% color change. In some implementations, percentage of wetting and color change may be determined by several techniques, including but not limited to by the human eye, photodetector (e.g., a photo camera), color reader, or spectrometer.

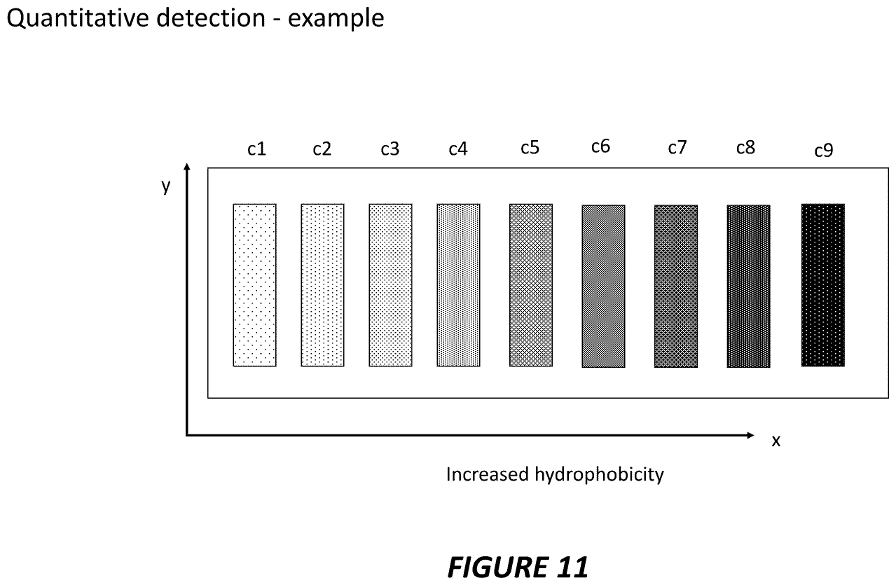

In some embodiments, the surface coverage of primers or the surface coverage of the hydrophobic molecule may not be homogeneous. For a non-limiting example as shown in FIG. 11, the hydrophobicity of the functional area shows a gradient distribution (e.g., increased hydrophobicity) along the longitudinal direction (e.g., x direction) of the sensor chip. Each zone has a different wetting threshold that is proportional to the concentration of the target analyte (e.g., c1, c2, . . . c9). The distribution of concentrations may be continuous or discrete along the x direction enabling a semi-quantitative or quantitative readout of the analyte concentration.

Percentage of wetting and color change may be determined by several techniques, including but not limited to by the human eye, photodetector (e.g., a photo camera), color reader, or spectrometer. The chip could be modified to fit existing systems with capable imaging and processing software to determine the quantitative detection. Thus, while instrumentation is not needed for a qualitative identification of the presence of a target analyte (e.g., a surface with greater than 5-10% wetting), some instrumentation may be used for subsequent or additional quantitative analysis of the chip.

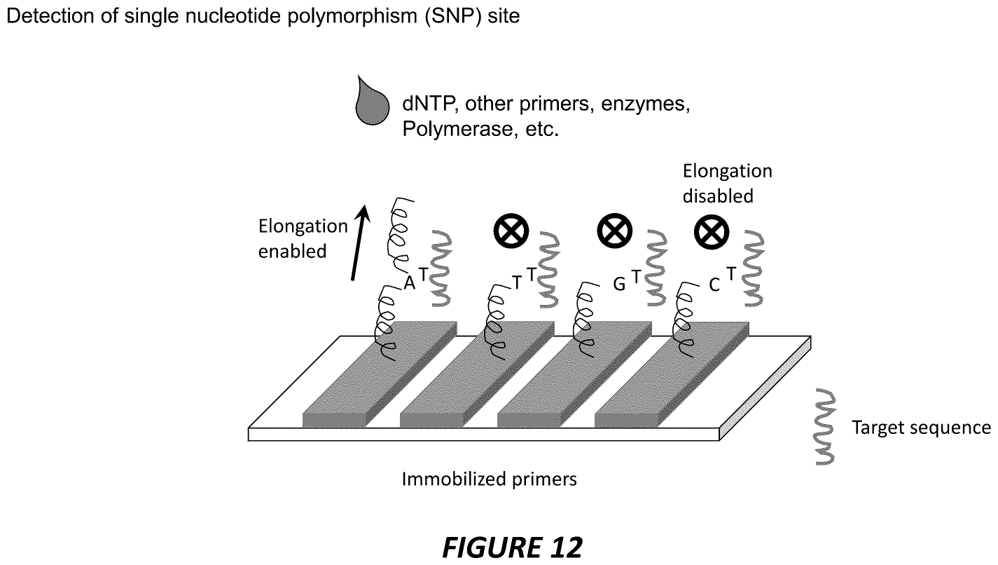

In some embodiments, the sensor may be modified for detection of single nucleotide polymorphism (SNP). As shown in FIG. 12, more than one different functional area (e.g., four areas) may be designed on the sensor surface. Four identical primers may be immobilized on the four functional areas with one end (e.g., 5′ end) and each primer may contain one variable base that is corresponding to the SNP site at the other end of the primer sequence (e.g., at 3′ end or elsewhere within the primer sequence). Among the four primers, only one primer may be fully complementary to the DNA target. For example, the primer with base A is fully complementary to the target with base T at the SNP site. When introducing dNTP, polymerase, and other enzymes to induce the solid-phase amplification, only the fully complementary primer with the target sequence is able to extend and induce a color change (and/or change in wetting state) in that primer-specific area. In some other implementations, multiple nucleotide polymorphism may be detected by repeating the detection scheme of the SNP for each nucleotide variant.

Different immobilization techniques may be used to immobilize primers, for a non-limiting example, a drop casting method may be used to physically attach the primers to the sensor surface at different locations. Alternatively, the drop casting method may be used along with a click chemistry reaction to form chemical bonding between the primers and different target-specific areas across the sensor surface. For example, primers may be modified with a 5′ hexynyl group that reacts with an azido functionalized sensor surface via a CuAAC click chemistry reaction. In some implementation, primers may be immobilized via EDC-NHS Carbodiimide cross-linking. Different primers may be immobilized on a specific area across the sensor surface by applying a photomask so that each type of aforementioned primer containing one variable base may be spatially separate and generate a unique color pattern when SNP or MVP is present. The color change and/or color change pattern may be directly visualized and quantified by naked human eyes or with the assistance of an image/spectrum recording device (e.g., a photo camera, an optical spectrometer, color reader, etc.).

### Example Methods of Manufacturing the Integrated Molecular Sensor Device

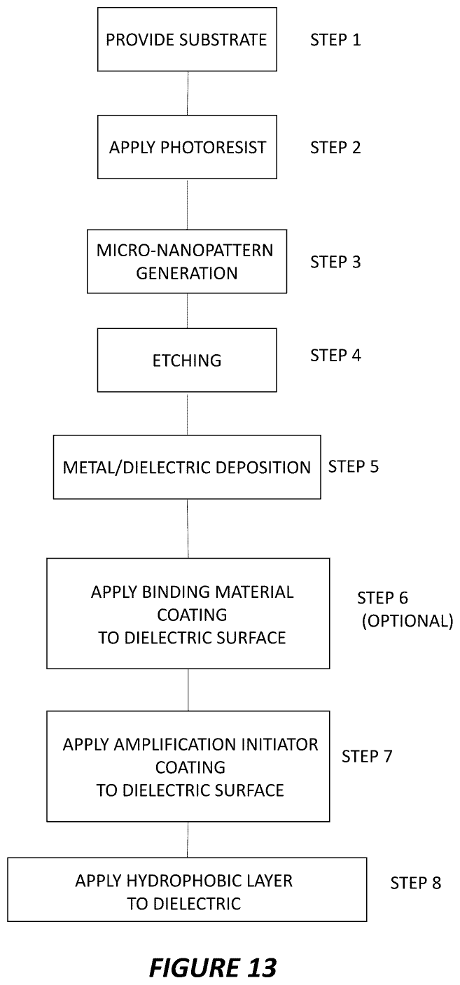

Having described a number of embodiments of sensing devices for detecting a target analyte of interest in a fluid (e.g., a liquid), example methods of producing wetting-based colorimetric sensing devices will now be described. In some embodiments, with reference to FIG. 13, after providing a base substrate (e.g., STEP 1) and applying a photoresist layer (e.g., PMMA, SU8, and so forth) with a thickness between 0.1 micrometer and 10 micrometers to the substrate (e.g., STEP 2), a nanopattern with predetermined geometry may be generated and transferred to the photoresist layer, for example, via photolithography, electron-beam lithography (EBL), and the like (e.g., STEP 3).

Once the micropattern or nanopattern has been added to the substrate, the micropattern or nanopattern may be etched e.g., (STEP 4). For example, microstructures and/or nanostructures 520 may be formed after etching part of the photoresist layer or etching into the base substrate (e.g., STEP 4). In a next step, a thin layer (e.g., of about 0.1 nm to several hundred nanometers) of metal (e.g., platinum, gold, silver, aluminium, copper, tungsten, and the like, and combinations thereof) and/or a dielectric material (e.g., silicon dioxide, titanium dioxide, hafnium oxide, and the like, and combinations thereof) may be applied (e.g., STEP 5) on the top surfaces of the microstructures and/or nanostructures 520, as well as on the bottom surface of each microfluidic groove 560. Example methods of applying a metal and/or dielectric material may include, for the purposes of illustration rather than limitation, metal deposition, chemical vapor deposition (CVD), sputtering, three-dimensional nanoprinting, plasma-enhanced chemical vapor deposition (PECVD), physical vapor deposition (PVD), electroless plating, and so forth.

In a next step, optionally, a binding material layer 530 may be applied to the metal and/or dielectric surface (e.g., STEP 6). In some implementations the binding material layer 530 may include aptamers, MIPs, antibodies, a combination thereof, and the like. Subsequently, an amplification initiator coating may be applied to the surficial walls or part of the surficial walls of the transducer 510 (e.g., STEP 7). Finally, a hydrophobic coating may be applied to the surficial walls or part of the surficial walls of the transducer 510 (e.g., STEP 8). Alternatively, the hydrophobic coating may be applied to the surficial walls or part of the surficial walls of the transducer 510 before applying the binding materials 530.

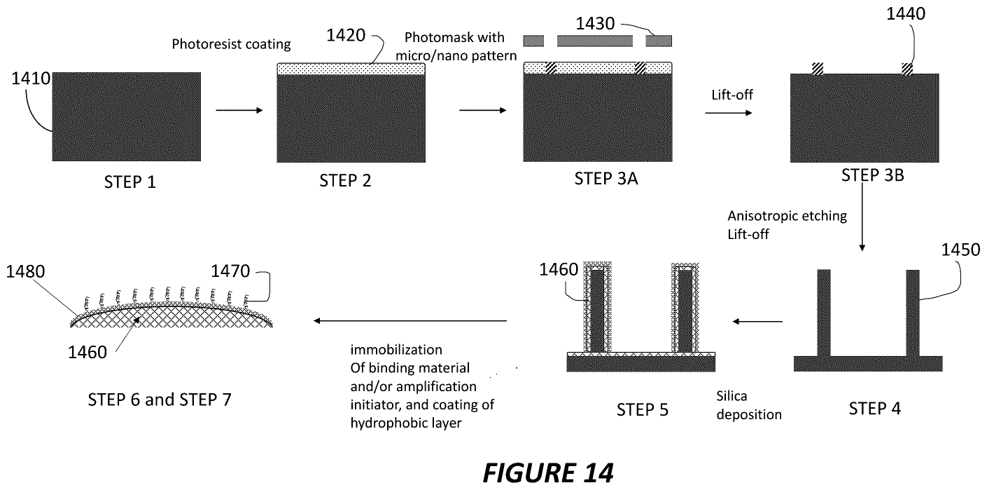

Referring to FIG. 14, a first example method of producing a silicon-based colorimetric sensor is shown. In some applications, in a first step, the base substrate 1410 (e.g., silicon or the like) is provided (e.g., STEP 1). Subsequently, a desired thickness of a photoresist layer 1420 may be applied (e.g., via spin-coating) on the surface of the silicon substrate 1410 (e.g., STEP 2). A photomask 1430 having a predetermined pattern (e.g., a micro-pattern, a nano-pattern, and the like) may be used to transfer the designed pattern 1440 onto a negative tone photoresist (e.g., a negative tone PMMA, SU8 photoresist, and the like) (e.g., STEP 3A, 3B). Design parameters may include, for the purpose of illustration rather than limitation: the shape of the microstructures and/or nanostructures 520; the size (e.g., diameter) of the microstructures and/or nanostructures 520; the periodicity between the microstructures and/or nanostructures 520; and so forth.

Isotropic etching may be applied to the patterned surface (e.g., STEP 4) to remove the portions of the base substrate 1410 that are not disposed immediately beneath the design pattern 1440. Example methods of etching include, for the purpose of illustration rather than limitation: electrochemical etching, wet-etching, dry-etching, and laser-induced etching. For example, in some variations, deep reactive ion etching, which can involve either a wet- or a dry-etching process, may be employed to form the microstructures and/or nanostructures 1450 in the base substrate 1410 using the patterned photoresist as an etching mask. In some variations, a Bosch process may be used to achieve high aspect ratio microstructures and/or nanostructures 1450 with roughness on the sidewalls of microstructures and/or nanostructures 1450. As another non-limiting example, a high-energy laser may be employed in a laser-induced etching process to form precise microstructures and/or nanostructures 1450 in the base substrate 1410 by etching away the material in the base substrate 1410.

A silica, thin film 1460 having a desired grain size (e.g., grains having a nanometre scale) may then be deposited onto the surface of the microstructures (and/or nanostructures) 1450 (e.g., via a PECVD process) (e.g., STEP 5). In some variations, silica nanoparticles may also be deposited to the surface of microstructures and/or nanostructures 1450. The silica nanoparticles may be crosslinked with each other and covalently grafted to the microstructure surface 1450 to enable a micro-nano hierarchical structure. Furthermore, the silica nanoparticles may be coated with an additional third-rank of molecular layers (e.g., a polymer layer with controlled thickness) to produce a micro-nano-angstrom hierarchical structure. The thickness of the silica thin film 1460 may be in the range of about 0.1 nm to several hundred of micrometers depending on the dimensions of the microstructures and/or nanostructures 1450.

A binding material 1470 (e.g., aptamers, MIPs, antibodies, and the like) and a hydrophobic layer 1480 (e.g., a fluorinated silane molecule, and the like) may then be applied to the surficial walls of the hierarchical structure. The order of the binding materials 1470 and the hydrophobic layer 1480 may be swapped depending on the immobilization method to be used. The hydrophobic coating 1480 may be applied in a variety of manners. For example, where the hydrophobic coating 1480 includes silane molecules, the hydrophobic coating 1480 may be applied via vapor phase deposition. As additional examples, where the hydrophobic coating 1480 includes dielectric materials (e.g., titanium dioxide, silicon dioxide, hafnium oxide, and the like), the hydrophobic coating 1480 may be applied via, for example: atomic layer deposition (ALD), plasma-enhanced chemical vapor deposition (PECVD), or physical vapor deposition (PVD). As yet another example, where the hydrophobic coating 1480 includes metallic materials (e.g., gold, silver, aluminum, copper, and the like), the hydrophobic coating 1480 may be applied via, e.g., an electron-beam evaporation process. In an example implementation where the binding material 1470 itself is hydrophobic, the additional hydrophobic coating 1480 may be unnecessary.

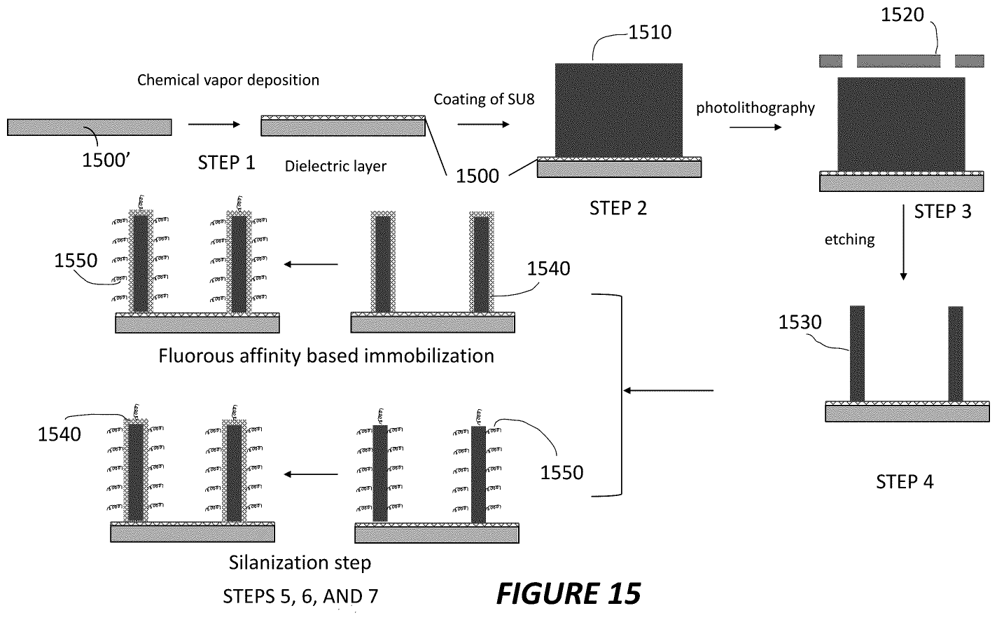

Referring to FIG. 15, a second example method of producing a SU8-based colorimetric sensor is shown. In a first step, a substrate 1500′ onto which a relatively thin layer of (e.g., dielectric, metallic, and the like) material (i.e., an indicator layer 1500) has been applied (e.g., via chemical vapor deposition) may be provided (STEP 1). The thin layer 1500 used as the extraction and indicator may include, but is not limited to: a thin layer of silicon dioxide 1500 (e.g., from 0.1 nm to several hundred micrometers) deposited on a silicon substrate 1500′, a thin layer of germanium 1500 (e.g., from 0.1 nm to several hundred micrometers) deposited on a gold substrate 1500′, and the like. Subsequently, a (e.g., SU8) photoresist layer 1510 may be applied to the top of the thin layer of indicator 1500 (STEP 2). In some variations, the thickness of the (e.g., SU8) photoresist layer 1510 may range between about 0.1 micrometer to 10 micrometers.

A photomask 1520 having a predetermined pattern (e.g., a micro-pattern, a nano-pattern, and the like) may be applied and used to transfer the desired pattern onto the SU8 photoresist 1510 (STEP 3). Design parameters may include, for the purpose of illustration rather than limitation: the size (e.g., diameter or other dimension) of the resulting microstructures and/or nanostructures 1530, the shape of the resulting microstructures and/or nanostructures 1530, the periodicity between the resulting microstructures and/or nanostructures 1530, and so forth.

Etching (e.g., isotropic etching) may be applied to the patterned surface (STEP 4) to remove the portions of the SU8 photoresist 1510 that are not disposed immediately beneath the design pattern 1520. Example methods of etching include, for the purpose of illustration and not limitation: electrochemical etching, wet-etching, dry-etching, and laser-induced etching. For example, deep reactive ion etching, which can involve either a wet- or a dry-etching process, may be employed to form the microstructures and/or nanostructures 1530 using the patterned photoresist 1520 as an etching mask. In some variations, a Bosch process may be used to achieve high aspect ratio microstructures and/or nanostructures 1530 with roughness on the sidewalls of the microstructures and/or nanostructures 1530.

After SU8 microstructures and/or nanostructures 1530 are produced, deposition may be performed to coat the surficial walls of SU8 microstructures and/or nanostructures 1530 with a thin layer 1540 (e.g., from 0.1 nm to several hundred micrometers) of silicon dioxide or other materials (STEP 5). In addition to silicon dioxide, the thin layer 1540 may include, for the purpose of illustration rather than limitation: dielectric materials, oxides, semiconductors, metals, combinations thereof, and the like. In some variations, the thin layer 1540 may be a continuous film (e.g., a smooth film with surface roughness less than 0.1 nm); while, in other variations, the thin layer 1540 may include isolated island structures (e.g., island nanostructures).

In a next step, a binding material 1550 (e.g., nucleic acids, aptamers, MIPs, antibodies, and the like) (STEP 6) and/or hydrophobic layer (STEP 7) may be coated on the SU8 microstructures and/or nanostructures 1530 and/or on the thin layer 1540. For example, in one variation, an immobilization method may involve a fluorous affinity-based interaction between a fluorous binding material 1550 (e.g., a primer) and a fluorous anchor molecule disposed on the surface of the microstructures and/or nanostructures 1530. In another example, a fluorinated silane 1540 may be applied after immobilization of binding materials 1550 onto the surface of the microstructures and/or nanostructures 1530.

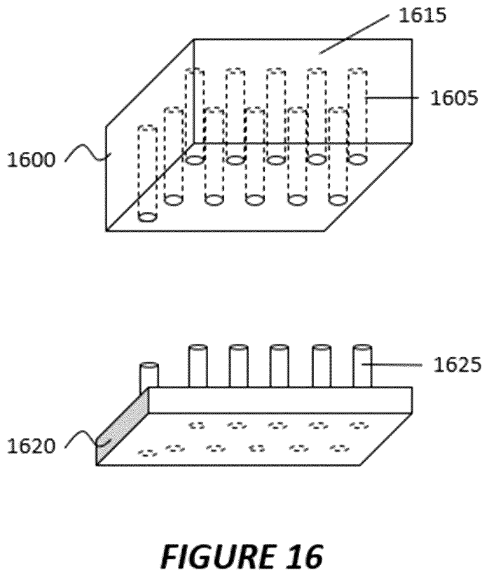

Referring now to FIG. 16, as an alternative to an etching process, in another example method of manufacture, sensors may be manufactured using a hole-like mold 1600. The hole-shaped mold 1600 is used as an example for the purpose of illustration rather than limitation. Those of ordinary skill in the art can appreciate that other types of molds (e.g., a pillar-like mold) may be used for imprinting to produce a hole-based structure. The hole-like mold 1600 may be made of, for example, silicone or some other suitable mold material such as silicon, polyethylene terephthalate (PET), a UV-curable resin, and the like. In some implementations, the mold 1600 is structured and arranged to include, on a bottom portion 1615 thereof, hollow portions 1605 with distances therebetween. The hollow portions 1605 are structured and arranged to provide a negative or mirrored image of a desired array of solid pillars 1625 in a base substrate 1620. As will be appreciated by those of ordinary skill in the art, although FIG. 16 shows a method in which the hollow portions 1605 are formed on the bottom portion 1615 of the mold 1600 and the bottom portion 1615 is pressed into a top surface of the base substrate 1620, the hollow portions 1605 may, instead, be formed on a top portion of the mold 1600 and the top portion pressed into the base substrate 1620.

The hollow portions 1605 of the mold 1600 may be configured to provide, in the base substrate 1620, a resulting pillar array 1625 that each has a desired size, shape, depth, periodicity, and so forth. Although the shape and size of each solid portion 1605 may be the same or substantially the same as one another, those of ordinary skill in the art can appreciate and understand that the hollow portions 1605 may instead be sized and shaped differently from one another so as to provide pillars 1625 of differing sizes, shapes, and depths, as well as of differing periodicity.

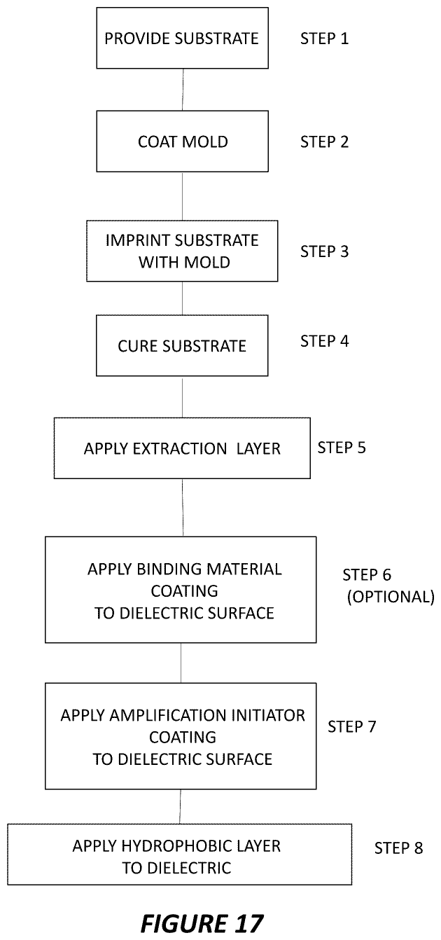

In accordance with an example method, and with reference now also to FIG. 17, after providing the base substrate 1620 and mold 1600 (STEP 1), the surfaces of the hollow portions 1605 of the mold 1600 may be coated (STEP 2) with a very thin layer of a releasing agent (e.g., fluorocarbon, fluorosilane, polybenzoxazine, combinations thereof, and the like) to facilitate removal of the mold 1600 from the resulting array of pillars 1625. The very thin layer or coating of the releasing agent can be a self-assembled monolayer (SAM) or multiple layers with a thickness from less than about one (1) Angstrom to about 10 nm. The base substrate 1620 may then be imprinted with the mold 1600 (STEP 3).

Following the imprinting of the pillars 1625 in the base substrate 1620 and depending on the material used to manufacture the base substrate 1620, the imprinted substrate 1620 may be cured (STEP 4), for example, via photo—(e.g., using ultraviolet (UV) light) or thermal-initiated polymerization.

In a next step, depending on the structural color generation mechanism and extraction menchanism, a thin layer (e.g., of about 0.1 nm to several hundred micrometers) of dielectric (e.g., silicon dioxide, titanium dioxide, hafnium oxide, and the like, and combinations thereof) and/or metal (e.g., platinum, gold, silver, aluminium, copper, tungsten, combinations thereof, and the like) may be applied (STEP 5) as an extraction layer on the top surface of the base substrate 1620, as well as the surficial walls of pillars 1625. Example methods of applying a dielectric or metal may include, for the purpose of illustration rather than limitation: metal deposition, chemical vapor deposition (CVD), sputtering, three-dimensional nanoprinting, plasma-enhanced chemical vapor deposition (PECVD), physical vapor deposition (PVD), electroless plating, sol-gel process and so forth. In some variations, the deposition may be on all or part of the surficial walls defining the pillars 1625. Deposition on the top surface of the base substrate 1620 may form a continuous metal film atop the substrate 1620.

In a next step, optionally, a binding material layer may be applied to the metal and/or dielectric surface (STEP 6, optional). In some implementations the binding material layer may include aptamers, MIPs, antibodies, a combination thereof, and the like. Binding materials (e.g., aptamers) may be applied to the top of the extraction layer (e.g., silica) as a thin (e.g., 0.1 nm to 1000 nm thick) coating. Example methods of applying binding materials to the top of the extraction layer may include, for the purpose of illustration rather than limitation: spin-coating, dip-coating, covalently binding, and the like. Subsequently, an amplification initiator coating may be applied to the surficial walls or part of the surficial walls of the pillar-like transducer 1625 (STEP 7). Finally, a hydrophobic coating may be applied to the surficial walls or part of the surficial walls of the pillar-like transducer 1625 (STEP 8). Alternatively, the hydrophobic coating may be applied to the surficial walls or part of the surficial walls of the pillar-like transducer 1625 before applying the binding materials.

### Example Binding Materials/Amplification Initiator and Methods of Attachment to the Sensors

In some implementations, the binding material 530 may be an oligonucleotide, for example, an aptamer. In some applications, the binding material 530 may also be an amplification initiator (e.g., a primer). In some implementations, the binding material 530 may be applied first to the surface of the pillar-like transducer and an amplification initiator (e.g., a primer) may be applied separately in the next step. In some other applications, the binding material 530 may be co-immobilized along with an amplification initiator to the surface of the pillar-like transducer. In the following text, we use “oligonucleotide(s)” or “oligo(s)” as a non-limiting example to include both cases of binding materials and amplification initiators. The concentration of oligos used in the conjugation process determines the density of oligos immobilized on the sensor surface. The concentration of oligos may be in the range from, e.g., 1 femtoMolar to 1 milliMolar, although other ranges are possible. Aptamers are single stranded oligonucleotide molecules that bind a specific target molecule. Aptamers with specificity for a target molecule are identified by screening oligonucleotide libraries using a process called SELEX (systematic evolution of ligands by exponential enrichment). The terminal functional groups of an oligonucleotide may be modified to allow the attachment and coating of an oligo to a colorimetric sensor surface. Although oligos may be bound to the surface via covalent bonds, those of ordinary skill in the art can appreciate that oligos may also be bound to the sensor surfaces via other types of chemical interaction, including but not limited to, non-covalent interactions, ionic bonds, van der Waals forces, electrostatic forces, hydrogen bonding, fluorous affinity, and Pi-Pi stacking interactions.

In some applications, the aptamer oligos for use as described herein (e.g., as a binding material, layer, and/or coating) may generally be manufactured by oligonucleotide synthesis using phosphoramidite chemistry. To obtain the desired aptamer, in this example, the building blocks are sequentially inserted into the growing oligonucleotide chain in the order of the aptamer sequence from the 3′ to 5′ direction. Typical (although non-limiting) numbers of nucleotides may be selected between 1 nucleotide and 200 nucleotides, between 10 nucleotides and 100 nucleotides, or between 30 and 80 nucleotides. Typical (although non-limiting) building blocks include, for the purpose of illustration and not limitation, nucleoside phosphoramidites, non-nucleoside phosphoramidites, and the like. In some implementations, the aptamer may be chemically modified at either its 5′ or 3′ end with specific functional groups that facilitate the crosslinking to the surface of the transducer 510 (e.g., amine modification).

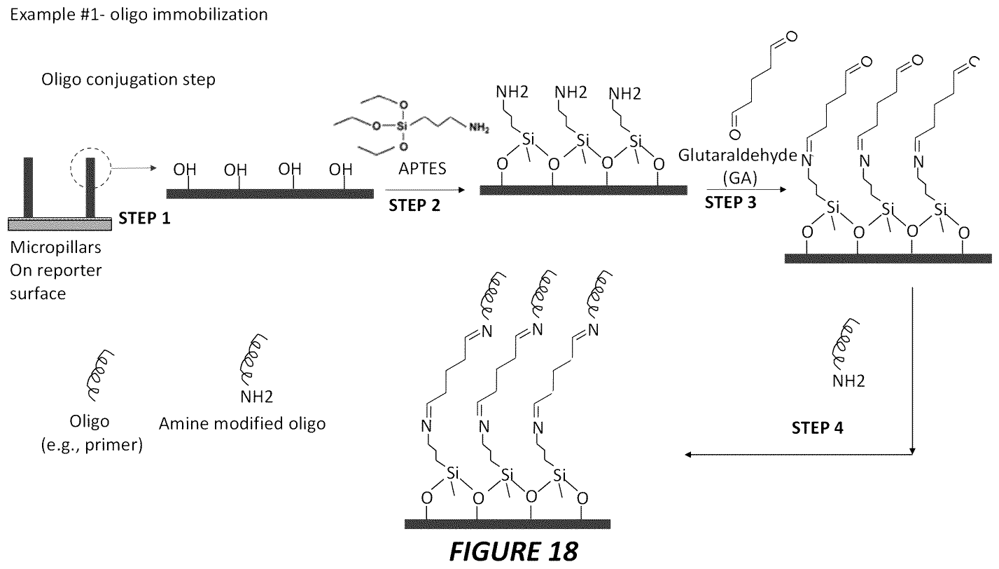

FIG. 18 shows an example scheme for conjugating an aminated oligo to a surface via glutaraldehyde crosslinking in a 4-step process. It will be appreciated after reading the present disclosure that these steps, as well as other steps described throughout, may be modified to achieve the desired result. STEP 1 may include an activation process to produce a hydroxyl-rich surface. The activation process may include, but is not limited to, an oxygen plasma treatment of the surface, a piranha solution mediated surface treatment, and the like. STEP 2 may include an amination process in which (3-Aminopropyl)triethoxysilane (APTES) is reacted with the hydroxyl-rich surface from STEP 1. This amination process may occur in either the solution phase or gaseous phase. For example, the amination may proceed at room temperature in a 2% APTES solution in toluene for one hour, or in APTES vapor phase within a sealed reactor at 150° C. Subsequently in STEP 3, the aminated surface may be reacted with one of the two carbonyl functional groups of a glutaraldehyde crosslinker to yield a carbonyl-rich surface. Then in STEP 4 the aminated oligo, which is made using a standard oligo modification, may be covalently linked to the carbonyl-rich surface of STEP 3 by, e.g., reacting with the carbonyl functional groups of the glutaraldehyde crosslinker. In some implementations, the concentration of aminated oligos may be in the range from, e.g., 1 femtoMolar to 1 milliMolar.

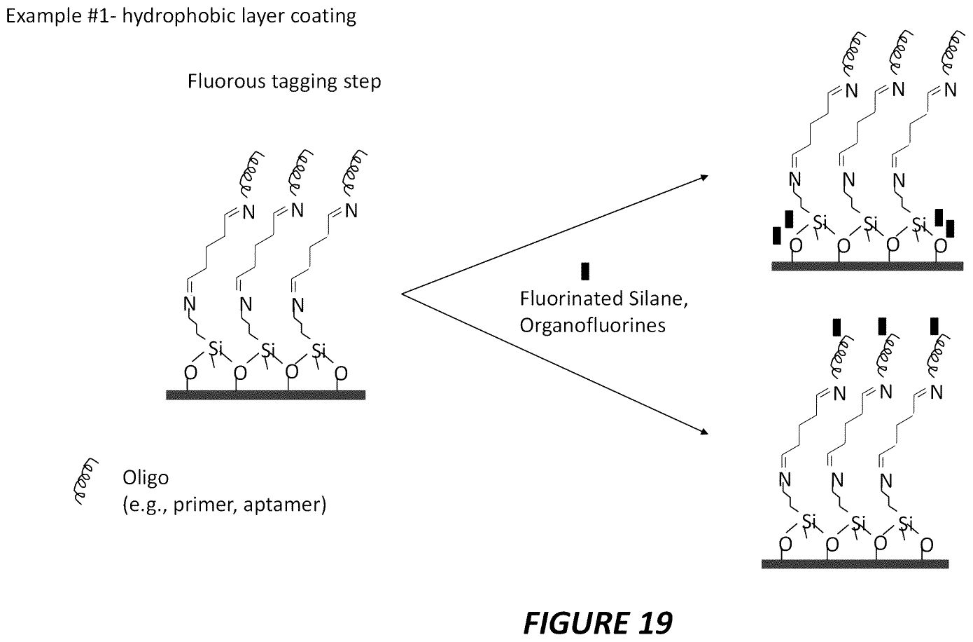

FIG. 19 depicts an example scheme for applying a hydrophobic coating to a sensor surface after attachment of the binding material and/or amplification initiator (e.g., aptamers, primers). Compounds with long alkyl chains, fluorinated silanes (e.g., 1H,1H,2H,2H-perfluorooctyltrichlorosilane, Octadecyltrichlorosilane, and the like), organofluorine compounds, perfluorocarbons, fluoropolymers, hydrofluorocarbons, fluorocarbenes, combinations thereof, and the like may be deposited on the sensor surface via liquid or vapor phase deposition. This hydrophobic coating may be formed at different locations of the sensor as shown in FIG. 19 and discussed throughout. These locations may include the surface of the transducer, the surface of the oligo, or a combination thereof. The hydrophobic molecular coating may be covalently grafted to the surface, physically adsorbed to the surface, or a combination thereof. It will be appreciated after reading the present disclosure that, in some embodiments, organofluorine gas (e.g., C₄F₈ as a non-limiting example) may be used in a plasma process to generate free radicals or fragments and deposited on the micro/nanopillar surface or covalently grafted to the oligo, or physically deposited on the surface via other molecular interactions than covalent bonding. It will be appreciated after reading the present disclosure that other techniques may be used to coat the sensor surface with these hydrophobic materials.

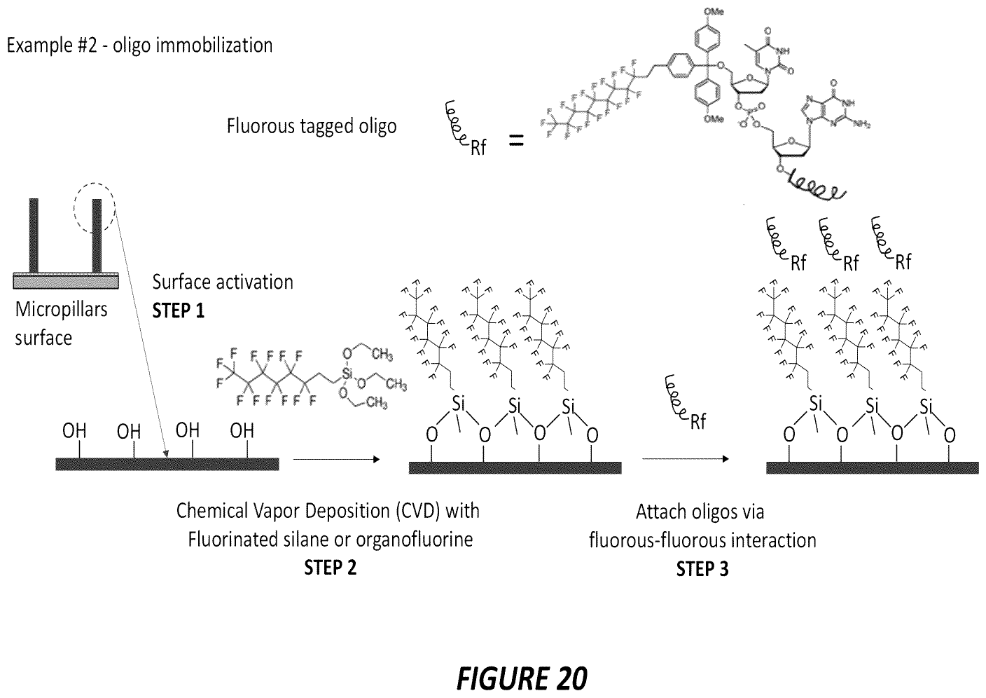

In some implementations, the oligo may be attached to the sensor surface using fluorous affinity. For example, FIG. 20 demonstrates a 3-step process to immobilize an oligo (e.g., an aptamer, a primer) to the surface. STEP 1 may include an activation process to produce a hydroxyl-rich surface. The activation process may include, but is not limited to, an oxygen plasma treatment of the surface, a piranha solution mediated surface treatment, and the like. In STEP 2, the activated transducer surface (e.g., a silicon dioxide surface) may be functionalized with fluorous molecules. For example, fluorous silane (e.g., 1H,1H,2H,2H-Perfluorooctyltriethoxysilane or the like) may be covalently grafted to the activated transducer surface via a chemical vapor deposition (CVD) process. It will be appreciated after reading the present disclosure that, in some embodiments, organofluorine gas (e.g., C₄F₈ as a non-limiting example) may be used in a plasma CVD process to generate free radicals or fragments and deposited on the micro/nanopillar transducer surface. In STEP 3, a fluorous tagged oligo may be attached to the fluorinated transducer surface via fluorous-fluorous interaction. Fluorous tagged oligos may be produced using well-known oligo modification procedures.

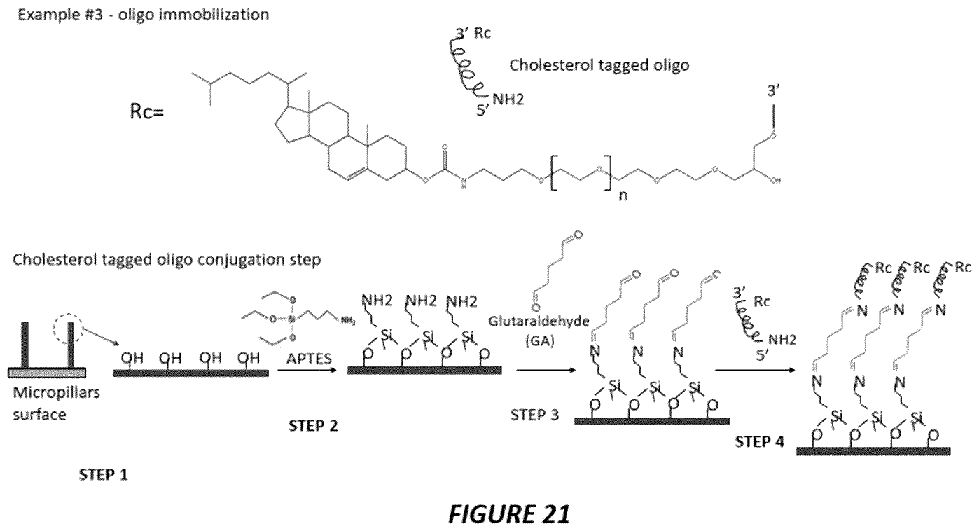

In some embodiments, in order to prepare an initial hydrophobic surface, the binding material may be produced using hydrophobic components (e.g., the binding material itself is hydrophobic; a hydrophilic binding material coated with a hydrophobic layer; or a combination thereof). FIG. 21 shows an example of how a hydrophobic lipid molecule, such as cholesterol, may be tagged to the 3′ (or 5′) end of an oligo while a primary amine group may be tagged to the 5′ (or 3′) end of the oligo. This tagged oligo can be made using a standard oligo modification process. A hydrophilic spacer may be introduced between the oligo and the hydrophobic cholesterol tag to fine tune the hydrophobicity to the desired level. For the purpose of example rather than limitation, the primary amine group is used to attach the aptamer to the sensor surface via glutaraldehyde crosslinking chemistry, as described above using a 4-step process. In some applications, a folding buffer may be used to fold the aptamer into the desired 3D structure at least when the aptamer is used as the binding material. Example folding buffers may include, but are not limited to, 137 mM NaCl, 2.7 mM KCl, 6.5 mM Na2HPO4, 1.8 mM NaH2PO4, 1.47 mM MgC12, 0.05% Tween-20, and 0.1% (w/v) bovine serum albumin (BSA).

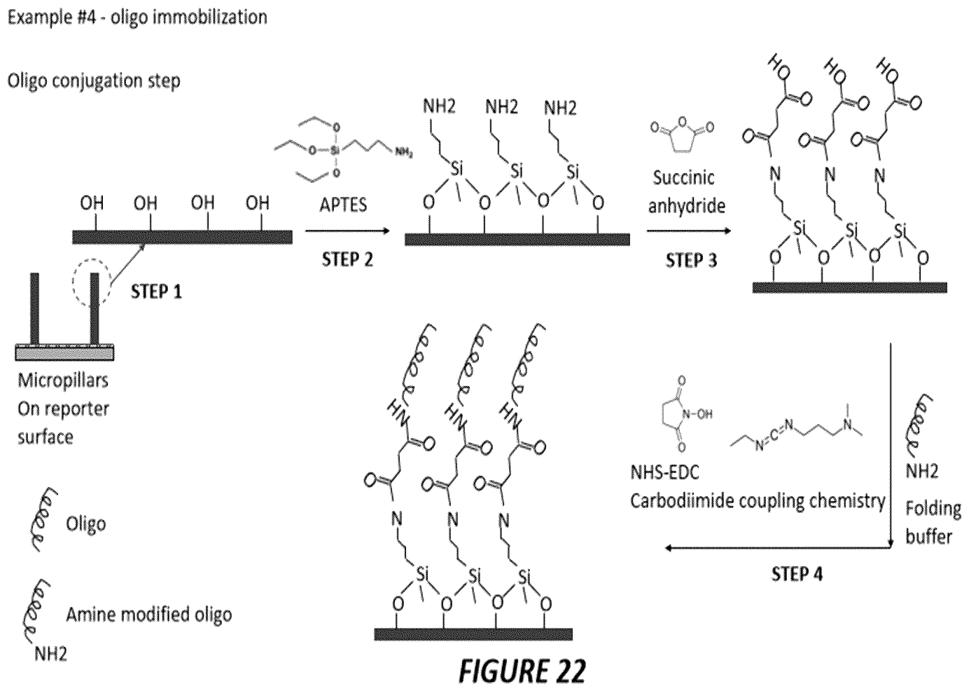

In some embodiments, amine modified oligos may be attached to the sensor surface via carbodiimide crosslinking chemistry. FIG. 22 demonstrates a four-step carboxyl-to-amine crosslinking process. STEP 1 may include an activation process to produce a hydroxyl-rich surface. The activation process may include, but is not limited to, an oxygen plasma treatment of the surface, a piranha solution mediated surface treatment, and the like. STEP 2 may include an amination process in which (3-Aminopropyl)triethoxysilane (APTES) is reacted with the hydroxyl-rich surface from STEP 1 to generate an amine-rich surface. This amination process may occur in either the solution phase or gaseous phase. For example, the amination may proceed at room temperature in a 2% APTES solution in toluene for one hour, or in APTES vapor phase within a sealed reactor at 150° C. Subsequently in STEP 3 the amine-rich surface may be converted into a carboxylic-rich surface via a ring opening amidation reaction of succinic anhydride with the primary amine groups on the sensor surface at room temperature. Finally, STEP 4 includes an EDC/NHS (1-Ethyl-3-[3-dimethylaminopropyl]carbodiimide hydrochloride/N-hydroxysuccinimide) coupling chemistry reaction, or in some variations, an EDC/sulfo-NHS reaction between an amine modified oligo and carboxylic-rich surface to prepare the modified sensor surface. In some implementations, the concentration of amine modified oligos may be in the range from 1 femtoMolar to 1 milliMolar, although other concentrations are possible.

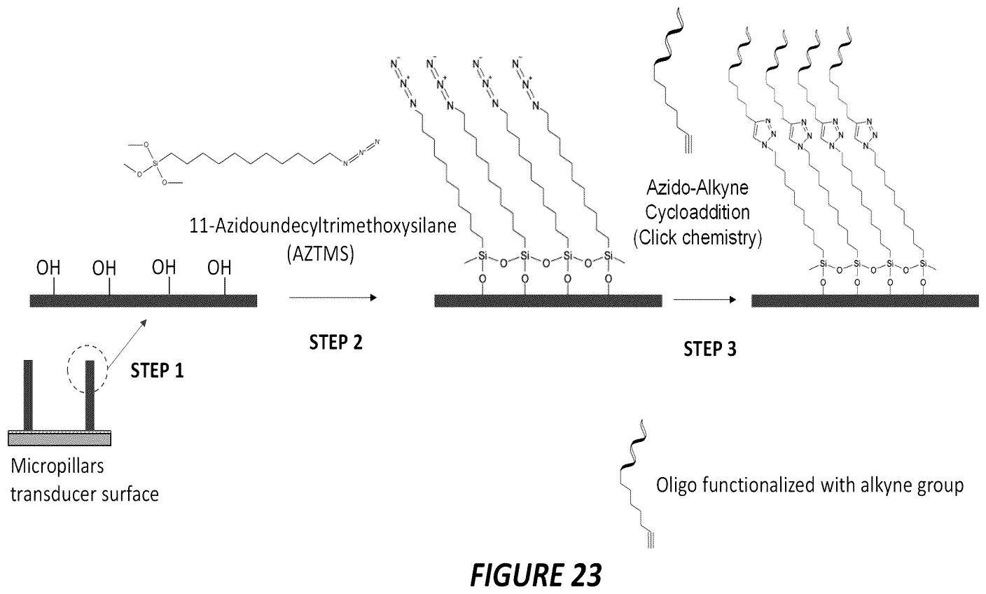

FIG. 23 depicts another example method for attaching oligos to the sensor surface via click chemistry reaction in three steps. For example, STEP 1 may include an activation process to produce a hydroxyl-rich surface, as described above. STEP 2 includes a vapor phase reaction between an azide-bearing clickable silane molecule (e.g., 11-Azidoundecyltrimethoxysilane) with the hydroxyl groups on the sensor surface to form an azide rich surface. STEP 3 includes a copper catalyzed azide alkyne cycloaddition (CuAAC) click chemistry reaction between the azide-rich surface and an alkyne modified oligo. The alkyne modified oligos can be prepared using a standard oligo modification (e.g., from Integrated DNA Technologies). The concentration of alkyne modified oligos may be in the range from 1 femtoMolar to 1 milliMolar. In some applications, other click chemistry attachments may be used including, but not limited to, Strain promoted alkyne-azide cycloaddition (SPAAC), also referred to as the Cu-free click reaction, and the Inverse electron demand Diels-Alder (IEDDA) click reaction. For example, in a SPAAC reaction, the azide modified aptamers, prepared by standard oligo modification, may be immobilized to the sensor surface via the reaction between the azide group and a dibenzocyclooctyne (DBCO) functionalized sensor surface. The DBCO surface may be produced from the reaction between a DBCO-NHS ester with an amine-rich surface, as described above.

The corresponding structures, materials, acts, and equivalents (e.g., of all means or step plus function elements) that may be in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications, variations, substitutions, and any combinations thereof will be apparent to those of ordinary skill in the art without departing from the scope and spirit of the disclosure. The implementation(s) were chosen and described in order to explain the principles of the disclosure and the practical application, and to enable others of ordinary skill in the art to understand the disclosure for various implementation(s) with various modifications and/or any combinations of implementation(s) as are suited to the particular use contemplated.

Having thus described the disclosure of the present application in detail and by reference to implementation(s) thereof, it will be apparent that modifications, variations, and any combinations of implementation(s) (including any modifications, variations, substitutions, and combinations thereof) are possible without departing from the scope of the disclosure defined in the appended claims.

What is claimed is:

1. A sensor chip for detecting an analyte of interest, the sensor chip comprising:
a structure comprising a plurality of walls that define a plurality of air gaps in the structure, wherein each wall of the plurality of walls includes a plurality of surfaces;
a functional layer, wherein the functional layer is coated on the plurality of walls, wherein the functional layer comprises an extraction component to extract an analyte of interest;
at least one amplification initiator to amplify the analyte of interest after extraction; and
a material coating the plurality of walls providing an initial surface energy for at least a portion of the plurality of surfaces of the plurality of walls,
wherein the initial surface energy of at least the portion of the plurality of surfaces of the plurality of walls provided by the material coating changes when the analyte of interest is present.

2. The sensor of claim 1, wherein the functional layer further comprises a binding material to bind to the analyte of interest after amplification in a solution phase.

3. The sensor of claim 2, wherein the change in the initial surface energy is due to the binding material binding amplicons in solution on at least the portion of the plurality of surfaces of the plurality of walls, preventing a fluid sample comprising the analyte of interest from exiting the plurality of air gaps.

4. The sensor of claim 1, wherein the change in the initial surface energy is due to amplicons forming on at least the portion of the plurality of surfaces of the plurality of walls preventing a fluid sample comprising the analyte of interest from exiting the plurality of air gaps after amplification on a solid phase.

5. The sensor of claim 1, wherein multiple amplification initiators are immobilized on at least the portion of the plurality of walls in spatially separated zones to enable multiplexing detection.

6. The sensor of claim 1, wherein a percentage of the initial surface energy change quantitatively determines a concentration of the analyte of interest.

7. The sensor of claim 6, wherein each amplification initiator has a variable base and a matching base that initiates an elongation of the each amplification initiator, producing the percentage of the initial surface energy change to indicate a single nucleotide polymorphism (SNP) site.

8. The sensor of claim 1, wherein the plurality of walls coated with the functional layer are fabricated from a micropillar array, a nanopillar array, or a combination thereof

9. The sensor of claim 1, wherein extraction and amplification are conducted on the functional layer via a solution phase amplification mechanism, a solid phase amplification mechanism, or a combination thereof

10. The sensor of claim 1, wherein the analyte of interest is one of a DNA sequence, a RNA sequence, a single nucleotide polymorphism, or multiple nucleotide polymorphism.

11. The sensor of claim 1, wherein the analyte of interest is a complementary DNA (cDNA).

12. The sensor of claim 1, wherein an RNA target forms a heteroduplex with a trigger DNA to initiate amplification.

13. The sensor of claim 1, wherein the analyte of interest is a microRNA sequence where a splinted ligation extends a short microRNA sequence template.

14. The sensor of claim 1, wherein the analyte of interest is a microRNA sequence where a poly(A) tail is added to a short microRNA sequence template.

15. The sensor of claim 1, further comprising an additional component tagging an amplicon or a reverse primer to enhance the change of the initial surface energy on at least the portion of the plurality of surfaces of the plurality of walls.

16. The sensor of claim 1, wherein the analyte of interest is amplified by loop-mediated isothermal amplification (LAMP), helicase dependent amplification (HDA), rolling circle amplification (RCA), recombinase polymerase amplification (RPA), strand-displacement amplification (SDA), nucleic acid sequence-based amplification (NASBA), or exponential amplification reaction (EXPAR).

17. A method of detecting an analyte of interest in a sample, the method comprising:
contacting the sensor of claim 1 with a sample;
extracting the analyte of interest with the functional layer;
amplifying the analyte of interest with the functional layer;
changing the initial surface energy of at least the portion of the plurality of surfaces of the plurality of walls when the analyte of interest is present; and
transitioning between a first mode and a second mode based upon, at least in part, the initial surface energy change.

18. The method of claim 17, wherein the functional layer further comprises a binding material to bind to the analyte of interest after amplification in a solution phase.

19. The method of claim 18 further comprising preventing a fluid sample comprising the analyte of interest from exiting the plurality of air gaps based upon, at least in part, the change in the initial surface energy due to the binding material binding amplicons in solution on at least the portion of the plurality of surfaces of the plurality of walls.

20. The method of claim 17 further comprising preventing a fluid sample comprising the analyte of interest from exiting the plurality of air gaps after amplification in a solid phase based upon, at least in part, the change in the initial surface energy due to amplicons formed on at least the portion of the plurality of surfaces of the plurality of walls.

21. The method of claim 17 further comprising immobilizing multiple amplification initiators on at least the portion of the plurality of walls in spatially separated zones to enable multiplexing detection.

22. The method of claim 17 further comprising quantitatively determining a concentration of the analyte of interest based upon, at least in part, a percentage of the initial surface energy change.

23. The method of claim 22, wherein each amplification initiator has a variable base and a matching base that initiates an elongation of the each amplification initiator that produces the percentage of the initial surface energy change to indicate a single nucleotide polymorphism (SNP) site.

24. The method of claim 17, wherein the plurality of walls coated with the functional layer are fabricated from a micropillar array or a nanopillar array.

25. The method of claim 17, wherein extraction and amplification are conducted on the functional layer via a solution phase amplification mechanism, a solid phase amplification mechanism, or a combination thereof.

26. The method of claim 17, wherein the analyte of interest is a DNA sequence, an RNA sequence, a single nucleotide polymorphism or a multiple nucleotide polymorphism.

27. The method of claim 17, wherein the analyte of interest is a complementary DNA (cDNA).

28. The method of claim 17, further comprising forming, by an RNA target, a heteroduplex with a trigger DNA to initiate amplification.

29. The method of claim 17 further comprising extending a short microRNA sequence template by a splinted ligation.

30. The method of claim 17 further comprising adding a poly(A) tail to a short microRNA sequence.

31. The method of claim 17 further comprising tagging an amplicon or a reverse primer with an additional component to enhance the change of the initial surface energy on at least the portion of the plurality of surfaces of the plurality of walls.

32. The method of claim 17, wherein the analyte of interest is amplified by loop-mediated isothermal amplification (LAMP), helicase dependent amplification (HDA), rolling circle amplification (RCA), recombinase polymerase amplification (RPA), strand-displacement amplification (SDA), nucleic acid sequence-based amplification (NASBA), or exponential amplification reaction (EXPAR).

33. The method of claim 17, wherein the first mode is a Cassie mode and the second mode is a Wenzel mode.

34. The method of claim 17, wherein the first mode is a slippery Wenzel mode and the second mode is a sticky Wenzel mode.
